(19) United States
(12) Patent Application Publication
(10) Pub. No.: US 2026/0042801 A1
(43) Pub. Date: Feb. 12, 2026

(54) COMPOSITIONS AND METHODS FOR DYE-BOUND CYCLIZED PEPTIDES FOR MEDICAL USE

(71) Applicant: The Texas A&M University System, College Station, TX (US)

(72) Inventors: Arthur SEFIANI, College Station, TX (US); Kevin BURGESS, College Station, TX (US); Cedric G. GEOFFROY, College Station, TX (US)

(21) Appl. No.: 19/100,885

(22) PCT Filed: Aug. 4, 2023
(86) PCT No.: PCT/US2023/071729

§ 371 Date: Feb. 3, 2025

Related U.S. Application Data
(60) Provisional application No. 63/395,491, filed on Aug. 5, 2022.

(51) Int. Cl. C07K 7/64; A61K 38/00; A61P 25/28; C07K 5/12

(52) U.S. Cl. CPC C07K 7/64; A61P 25/28; C07K 5/126; A61K 38/00

(57) ABSTRACT

Embodiments of the disclosure include methods and compositions related to use of compound comprising a particular peptide linked to a dye. In specific embodiments, the peptide DIRG is linked to the BODIPY dye. In specific embodiments, the compositions are utilized for treatment of neurological conditions and secondary pathologies related therewith, such as spinal cord injury and Alzheimer's Disease, as examples.

This application claims priority to U.S. Patent Application Ser. No. 63/395,491, filed Aug. 5, 2022, which is incorporated by reference herein in its entirety.

This invention was made with government support under R01EY029695 awarded by the National Institutes of Health. The government has certain rights in the invention.

### BACKGROUND

### I. Technical Field

This disclosure relates at least to the fields of cell biology, neurology, molecular biology, small molecules, and medicine

### II. Background

Neurotrauma and neurodegenerative diseases are devastating to both patients and the economy. One example of each, spinal cord injuries (SCI) and Alzheimer's disease (AD), are addressed herein.

There are over 1.4 million people living with paralysis associated with spinal cord injuries with 17,000 new patients each year in the United States alone. Besides placing extreme hardships on patients and their families, the cost of care for each traumatic spinal cord injury (SCI) patient is up to $1 million the year of injury and $5 million throughout the patient's lifetime¹. SCI is costing our economy over $40 billion annually². Neurons responsible for controlling movement extend axons into the spinal cord which, along with local spinal neurons, degenerate after SCI, leading to paralysis. SCI also induces cognitive dysfunction; 64% of people living with SCI develop cognitive impairments including diminished visual and working memory, which is 13-fold greater than the general population³⁻⁷, and impaired ability to learn new tasks and create new memories with a decrease in information processing speed and verbal fluency⁸. To date, there are no FDA approved treatments for reversing the loss of locomotor function or improving cognitive capacity after SCI.

Methylprednisolone, a corticosteroid, is the only FDA approved drug for SCI designed to mitigate secondary inflammatory pathologies such as swelling in the spinal cord. Methylprednisolone does not promote locomotor recovery⁹ and can be deleterious when administered >8 hours post injury¹⁰. Its efficacy is controversial even when administered within 8 hours⁹. Spinal decompression is an approved surgical operation conducted on SCI patients right after injury, which is also designed to reduce swelling and pressure on the spinal cord to mitigate secondary pathologies¹¹. Although both strategies can improve overall neurological outcome, neither strategy is designed to improve locomotor function. To date, there are no FDA-approved treatments aimed at improving functional locomotor recovery or cognition after SCI.

SCI not only affects motor and sensory function within the central nervous system, but also disrupts the peripheral neural circuitry and signals to vital organs in the body, resulting in severe long-term complications outside of the central nervous system. SCI has major effects on body composition and metabolism, which leads to a variety of risk factors including obesity, lower limbs skeletal muscle atrophy, decreased daily energy expenditure, changes in glucose-insulin homeostasis, and cardiovascular disease. Shortly after SCI, body weight is significantly decreased due to the reduction in lean body mass and fat depots. Ultrasound imaging demonstrated approximately 80% of chronic SCI patients exhibited liver abnormality. Magnetic resonance imaging (MRI) demonstrated increased liver adiposity as a result of SCI which impacts the metabolic profile. SCI also induces hepatic changes including increased lipid infiltration and inflammation in the liver. The systemic inflammation and increased inflammatory cytokines in the liver following SCI has high potential to induce liver dysfunction. SCI disrupts the innervation of the heart, causing cardiovascular autonomic dysfunction, leading to blood pressure and heart rate dysregulation resulting in cardiovascular pathologies¹². Furthermore, there is a 539% increase in infection, 280% increase in respiratory disease, and 139% increase in gastrointestinal-renal related fatalities per capita for people living with SCI relative to the average per capita fatality rates in the United States¹³. Therefore, treatments promoting neurological recovery may indirectly affect peripheral neural circuitry and other organs, including liver, heart, kidneys, lungs, and gastrointestinal tract.

Alzheimer's disease (AD) is a progressive disorder that is characterized by the aggregation of amyloid-β plaques (Aβ) and neurofibrillary tangles (NFT), synaptic loss, and cognitive decline^{14,15}. AD impacts over 6 million Americans¹⁶ costing the U.S. economy $305 billion annually¹⁷. Despite significant advancements in the functional understanding of AD progression in recent years and the financial incentives of developing AD therapeutics, which has a $9.5 billion market value and 17.5% compound annual growth rate (CAGR)¹⁸, there is still no cure for the disease. Current treatments such as NMDA antagonists and cholinesterase inhibitors are FDA-approved for slowing down cognitive deficits, however, their efficacy is modest to nonexistent while inducing serious adverse events¹⁹⁻²¹ which significantly reduce patients' quality of life. This is a major issue because many patients are not diagnosed early enough to be positively impacted by these FDA-approved drugs^{22,23}. Even if diagnosed, only 35-45% of patients or their caregivers are even informed about the diagnosis²⁴. Therefore, in most cases, there is a dire need for therapeutics that halt progression of AD and promote neurological recovery rather than prevent onset.

The tropomyosin receptor kinase (Trk) family has previously been targeted to provide functional recovery in several neurological disorders, such as cognitive impairment, AD, SCI, stroke, and Parkinson's disease⁵⁵⁻³⁰. Brain-derived neurotrophic factor (BDNF) and Neurotrophin (NT)-4 bind to TrkB, each mediating distinct neuronal funtions³¹. BDNF, which declines with age³², improves neuron survival, neuritogenesis, and neuronal plasticity^{33,34}. BDNF treatment reduces inflammation and promotes locomotor recovery in mouse SCI model³⁴⁻³⁶. With age, neurons are more reliant on NT-4 for survival³⁷, which enhances axon regenerative capabilities after nerve injury³⁸, improves learning memory³⁹, reduces neuroinflammation⁴⁰, and promotes recovery after stroke^{41,42}. Activation of TrkB has shown to reduce neuropathic pain, improve plasticity and cognitive function, and promote functional recovery after SCI^{36,43-46}. NT-3 binds to TrkC⁴⁷ which also decreases with age⁴⁸. NT-3 is vital for the survival of neurons^{49,50}, enhances myelination of regenerating axons in rodent SCI model⁵¹, augments regeneration of brain regions in non-human primate SCI model⁵², improves cognitive function⁵³, and promotes functional locomotor recovery^{54,55}. Therefore, activation of TrkB and TrkC is expected to promote both functional locomotor recovery and cognitive function in patients with locomotor and cognitive deficits. The translational challenge is TrkB/TrkC activating neurotrophins, such as BDNF and NT-3, are not therapeutically viable due to poor pharmacokinetic profiles including short half-lives and lacking CNS permeability^{56,57}. Synthetic TrkB/TrkC modulators can lack specificity and bioavailability, impeding their clinical potential. Thus, there is a clear unmet need for translatable, potent, and selective TrkB/TrkC modulator which can simultaneously address a number of therapeutically interesting pathophysiological processes.

### SUMMARY

Embodiments of the disclosure include methods and compositions for the treatment of a medical condition in which one or more dye-bound cyclized peptides and/or a functional derivative(s) thereof is useful to treat at least one symptom of the medical condition. In specific embodiments, the medical condition affects the central nervous system or peripheral nervous system. In some embodiments, the treatment with dye-bound cyclized peptide and/or a functional derivative thereof improves at least one symptom of the medical condition and also in certain embodiments leads to improvements for secondary pathologies associated with the medical condition. In some embodiments, dye-bound cyclized peptide and/or a functional derivative thereof improves a neurological disorder of any kind in an individual, and in specific embodiments also improves one or more secondary pathologies, such as liver fibrosis and/or heart atrophy, in the same individual. In particular embodiments, the dye-bound cyclized peptide(s) induces improvements in neurological recovery and may also lead to improvements in other secondary pathologies, such as liver fibrosis and heart atrophy, because of the dye-bound cyclized peptide directly promoting recovery of said organs, or indirectly by promoting overall health. In specific cases, the dye-bound cyclized peptide is 5c(i), as one example.

Embodiments of the disclosure include methods of treating a subject with a therapeutic composition, the method comprising a step of administering the therapeutic composition to the subject, wherein the therapeutic composition provides neuroprotection to the subject, modulates glial scar formation in the subject, treats neurotrauma in the subject, treats a neurodegenerative disease in the subject, or any combination thereof. In some embodiments, the therapeutic composition may comprise a peptide-based therapeutic composition. In specific embodiments, the peptide-based therapeutic composition may comprise any dye, including at least C-DIRG-C BODIPY® (fluorescent dye). In some cases, the C-DIRG-C BODIPY® (fluorescent dye) compound has the formula in FIG. 1. In particular embodiments, neuroprotection provided by 5c(i) or another one or more dye-bound cyclized peptides and/or a functional derivative(s) comprises an increase in neurite regeneration, an increase in neurite sprouting, an increase in axon regeneration, and/or an increase in axon sprouting. In certain embodiments, the neuroprotection comprises an increase in survival or viability of neurons and/or its neurites, axons, and/or dendrites, and/or neural cells involved in neural viability. In some embodiments, the modulation of glial scar formation provides wound closure of a spinal cord injury.

Embodiments of the disclosure include methods of treating a medical condition in an individual, comprising the step of administering a therapeutically effective amount of a compound of Formula I to the individual, or a functional derivative thereof, wherein the medical condition is a neurological disorder, fibrosis, liver disease, kidney disease, colon disease, a cardiac condition, a metabolic pathology, a muscle-associated disease, respiratory diseases, cancer, osteoporosis, bone loss, pain disorder (e.g., neuropathic pain), spleen disease, pancreas disease, pathological hormonal change(s), Peripheral Nerve Injury, a combination thereof, and so forth.

Because these compounds are bound by a dye (fluorescent or not, and fluorescent in the case of 5c(i)), other uses for these compounds could be as a tracking system for the compound itself (e.g., for mechanism of action, pharmacokinetic, and toxicity studies) and/or as a tracking system for the targets to which the compound binds. For example, in specific embodiments if the compound(s) bind to a specific protein, the compounds may be used in immunohistochemical assays to visualize the protein, or may be injected into live animals to visualize the movement of that protein therein. If that protein target of the compound is cancer-related, for example, the compounds could be used to visualize, track, and/or quantify cancer markers, such as for diagnostic purposes and/or monitoring of disease purposes (such as to monitor efficacy of one or more therapies in the individual).

Embodiments of the disclosure include methods of treating a neurological disorder, injury to the central or peripheral nervous system, and/or secondary pathology thereof, comprising administering to the individual a therapeutically effective amount of a composition comprising a compound of formula I as described elsewhere herein. In some embodiments, the neurological disorder is a neurodegenerative disease. In some embodiments, the neurological disorder and/or secondary pathology thereof comprises Alzheimer's Disease, multiple sclerosis, frontotemporal dementia, psychiatric disorders, Amyotrophic Lateral Sclerosis (ALS), ataxia, Bell's Palsy, brain tumor, cerebral aneurysm, encephalitis, epilepsy, seizures, Guillain-Barré Syndrome, dementia, migraine, Friedreich ataxia, Huntington's disease, Lewy body disease, Spinal muscular atrophy, or Parkinson's Disease. In specific embodiments, the injury to the central or peripheral nervous system comprises neurotrauma, such as neurotrauma that comprises concussion, traumatic brain injury, skull fracture, spinal column fracture, and/or spinal cord injury. Any compound encompassed herein may comprise exactly or at least the amino acids DIRG. In specific embodiments, the compound may comprise the amino acids CDIRGC.

Any compound utilized herein may include a Y group that is a rhodamine, fluorene, or BODIPY® dye, for example, or it may be an organic fragment that provides spacing between the two sulfur atoms as in BODIPY® dye±1 Angstrom. Such a fragment may comprise a chain with 5 atoms, in specific cases. In some embodiments, Y is not a dye. In certain embodiments, Y is

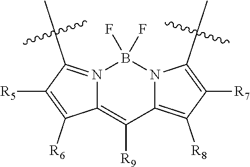

and in specific cases R₅, R₆, R₇, and R₈ are each independently H, halide, C₁-C₃ alkyl, or SO₃H or a salt thereof; R₅ and R₆, can optionally join to form a ring; R₇ and R₈ can optionally join to form a ring; and/or R₉ is H, halide, C₁-C₃ alkyl, or SO₃H or a salt thereof, or aryl.

In particular embodiments, an individual has a secondary pathology that comprises fibrosis (e.g., liver fibrosis), liver disease, kidney disease, colon disease, a cardiac condition, a metabolic pathology, a muscle-associated disease, respiratory diseases, and/or cancer.

Embodiments of the disclosure include methods of improving locomotor function, improving motility, improving long-term memory, improving cognition, reducing fibrosis, and/or reducing inflammation in an individual, comprising administering to the individual a composition comprising a compound of formula I as described elsewhere herein. In certain embodiments, the individual may have a neurological disorder, injury to the central or peripheral nervous system, and/or secondary pathology thereof. The neurological disorder may be a neurodegenerative disease. In specific embodiments, the neurological disorder and/or secondary pathology thereof comprises Alzheimer's Disease, multiple sclerosis, frontotemporal dementia, psychiatric disorders. Amyotrophic Lateral Sclerosis (ALS), ataxia, Bell's Palsy, brain tumor, cerebral aneurysm, encephalitis, epilepsy, seizures, Guillain-Barré Syndrome, dementia, migraine, Friedreich ataxia, Huntington's disease, Lewy body disease, Spinal muscular atrophy, or Parkinson's Disease. An injury to the central or peripheral nervous system may comprises neurotrauma, such as concussion, traumatic brain injury, skull fracture, spinal column fracture, and/or spinal cord injury.

In particular aspects of any composition encompassed herein, a compound may comprise, consist of, or consist essentially of the amino acids DIRG. The compound may comprise the amino acids CDIRGC in some instances.

In certain embodiments, the Y group of Formula I is a rhodamine, fluorene, or BODIPY® dye. In some cases, Y may be

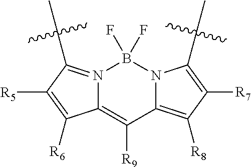

and R₅, R₆, R₇, and R₈ may each be independently H, halide, C₁-C₃ alkyl, or SO₃H or a salt thereof; R₅ and R₆, can optionally join to form a ring; R₇ and R₈ can optionally join to form a ring; and R₉ is H, halide, C₁-C₃ alkyl, or SO₃H or a salt thereof, or aryl.

In some cases, an individual may have a secondary pathology that comprises fibrosis (including liver fibrosis), liver disease, kidney disease, colon disease, a cardiac condition, a metabolic pathology, a muscle-associated disease, respiratory diseases, and/or cancer.

Embodiments of the disclosure include methods of monitoring location of a compound in vivo or ex vivo comprising directly or indirectly identifying a compound comprising formula I in the individual and/or a molecule to which the compound binds. The compound may comprise the amino acids DIRG exactly, or there may be additional amino acids. In some cases, the compound comprises the amino acids CDIRGC. The Y group may be a rhodamine, fluorene, or BODIPY® dye. In some embodiments, Y is

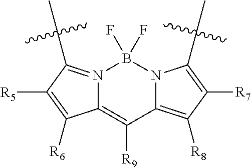

Wherein R₅, R₆, R₇, and R₈ are each independently H, halide, C₁-C₃ alkyl, or SO₃H or a salt thereof; R₅ and R₆, can optionally join to form a ring; R₇ and R₈ can optionally join to form a ring; and/or R₉ is H, halide, C₁-C₃ alkyl, or SO₃H or a salt thereof, or aryl.

In some embodiments, the method provides information about the mechanism of action, pharmacokinetic, and/or toxicity of one or more compounds, such as using an immunohistochemical assay. In some embodiments, the method comprises monitoring in a mammal the location of the compound or a molecule to which it binds, which may or may not be a cancer antigen or marker. Any individual may be known to have cancer, is suspected of having cancer, or is at risk for having cancer. In some embodiments, the method provides diagnostic and/or prognostic information for an individual suspected of having cancer or at risk for having cancer. In a specific embodiment, the method provides information about efficacy of therapy for an individual known to have cancer, suspected of having cancer, or at risk for having cancer.

Any method encompassed herein may be performed once for an individual or more than once for an individual. In specific cases, the method may monitor efficacy of a therapy over time for an individual. In some cases, the therapy is the compound itself, whereas in other cases the therapy is not the compound.

It is contemplated that any embodiment discussed in this specification can be implemented with respect to any method or composition of the disclosure, and vice versa. Furthermore, compositions of the invention can be used to achieve methods of the disclosure.

Other objects, features and advantages of the subject matter of the present disclosure will become apparent from the following detailed description. It should be understood, however, that the detailed description and the specific examples, while indicating specific embodiments of the disclosure, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Chemical structure of 5c(i).

FIG. 2: 5c(i) binds to TrkB and TrkC receptors. Transfected cell lines that are Trk positive cells (TrkB-HEK293 and TrkC-NIH/3T3) and non-transfected cell lines that are Trk negative cells (HEK293 and NIH/3T3) were seeded approximately 2,000 cells/well in a 96-well plate and incubated for 24 hours. The cells were treated with a serial dilution of compound (0, 10, 25, 50, 100, 200, 500, and 1000 nM) in serum-free media (SFM) with and without A) 0.6 nM of BDNF (TrkB-HEK293 and HEK293) and B) 0.2 nM of NT-3 (TrkC-NIH/3T3 and NIH/3T3) for 2.5 h. After that, the cells were washed with PBS once to remove unbound compounds and dissolved in 1% (w/v) aqueous sodium dodecyl sulfate. Cell-associated fluorescence was then determined by measuring the emission of the resulting solution upon λex (540/25 nm) and λem (620/40 nm) using Agilent BioTek Synergy H4 Hybrid Microplate Reader. Kd was calculated by GraphPad Prism 9 using Binding saturation (One site—Specific binding).

FIGS. 3A-3C: 5c(i) augments neuritogenesis and neuron survival. Primary adult cortical neurons from 6-month-old female mice were treated with 5c(i) for 2 days. 5c(i) was added at the time of cell plating. Cells were fixed 2 days after cell plating. 3A) Graph represents mean±SEM of percent change in neurite outgrowth per cell and number of surviving neurons. Representative images of 3B) Vehicle and 3C) 5000 nM 5c(i) treated groups. Linear regression was conducted to determine statistical significance. N=2-8. * (p<0.05) ** (p<0.01) *** (p<0.001) **** (p<0.0001) relative to vehicle control group. Scale bar=50 μm.

FIGS. 4A-4D: 5c(i) induces Synapsin I production and reduces C3 and GSK-3s expression from astrocytes promoting neuron survival and neurite growth. Primary adult cortical astrocytes from 6-month-old male mice were incubated with 5 μM 5c(i) for 3 days. Graph represents mean±SEM of percent change in fluorescent intensity (Synapsin I and C3) or chemiluminescence (GSK-3β) relative to Vehicle (4A and 4D). Images represent the 4B) Vehicle and 4C) 5 μM 5c(i) treated groups stained with Synapsin I and DAPI. D) Cortical astrocytes from 6-month-old male mice were plated and treated with 5c(i) or Vehicle for 2 days. Cells were then washed and cortical neurons from 6-month-old male mice were added on top of the astrocytes. Graph represents mean±SEM of percent change relative to Vehicle measured using immunocytochemistry. * (p<0.05) ** (p<0.01) *** (p<0.001) **** (p<0.0001). Scale bar=50 μm.

FIGS. 5A-5C: 5c(i) reduces injury size. Primary adult cortical astrocytes from 6-month-old female mice were scratched to form the injury model and immediately after were incubated with 5c(i) for 48 hours (5A). Graph represents mean t SEM. Images represent the 5B) Vehicle and 5C) 5000 nM 5c(i) treated groups stained with Phalloidin. Linear regression was conducted to determine statistical significance. N=10. Scale bar=500 μm.

FIG. 6: 5c(i) penetrates the blood-brain and -spinal cord barriers. 4-month-old uninjured female mouse was IP administered 60 mg/kg 5c(i). The mouse was euthanized 1 hour after 5c(i) administration and perfused with PFA. The spinal cord was dissected and sectioned sagittal at 25 μm and stained with NeuN and DAPI. The brain was dissected and sectioned coronal at 50 μm and stained with GFAP and DAPI. 5c(i) was identified through its intrinsic fluorescence. Scale bar=100 μm.

FIG. 7: Graphical representation of the activity of 5c(i) on neurons and astrocytes.

FIG. 8: Timeline of the preclinical in vivo animal study conducted as evidence of claims. Mice underwent baseline cognitive testing and blood collection immediately before SCI. Mice sustained an SCI at 8 weeks of age. SCI model: thoracic-8 contusion (50 kdyne impact) and compression (2—see dwell time), a severe contusion/compression model that accurately represents injuries seen in the clinic. 5c(i) (5 or 25 mg/kg/day) or Vehicle were administered IP daily starting 4 hours post injury to reflect the delay found in the clinic. Locomotor behavioral testing (BMS and Rotarod) were conducted weekly for 7 weeks. 7 weeks after injury, the mice underwent cognitive behavioral testing (NOR and Y-maze) and blood collection once again. Mice were euthanized 7 weeks after sustaining an SCI and underwent perfusion and tissue preparations for further analysis.

FIGS. 9A-9C: 5c(i) reverses paralysis and improves survival. 8-week-old male mice with a severe T8 contusion/compression SCI were injected IP daily with 5c(i) for 7 weeks (5 and 25 mg/kg/day), during which, behavioral testing was conducted. 9A) BMS Score and 9B) BMS Sub Score were recorded and graphed in relation to time after injury. 9C) Percent of mice surviving at 49 days post injury. SHAM mice have T7-T9 laminectomies only and are treated with vehicle only. N=6-8. Graphs show mean and SEM. The color of the following symbols # (p<0.1) * (p<0.05) ** (p<0.01) *** (p<0.001) **** (p<0.0001) represents significant difference between that cohort and vehicle (injured). Student's T-test.

FIGS. 10A-10C: 5c(i) improves memory function. 8-week-old male mice with a severe T8 contusion/compression SCI were injected IP daily with 5c(i) for 7 weeks (5 and 25 mg/kg/day). 7 weeks post injury, NOR was conducted. 10A) The total number of independent visits to novel object. 10B) Latency to visit novel object. 10C) The ratio of time spent with objects (novel/familiar ratio). SHAM mice have T7-T9 laminectomies only and are treated with vehicle only. N=6-8. Graphs show mean and SEM. # (p<0.1) * (p<0.05) ** (p<0.01) *** (p<0.001) **** (p<0.0001). Student's T-test.

FIGS. 11A-11E: Histological analysis of spinal cords after 5c(i) treatment. 8-week-old male mice with a severe T8 contusion/compression SCI were injected IP daily with 5c(i) for 7 weeks (5 and 25 mg/kg/day). 7 weeks post injury, spinal cords were sectioned sagittal to conduct histological analyses. Value of 11A) change in intensity of 5-HT, average fluorescence intensity of 11B) IBA1 and 11C) CD68, and number of 11D) CD68+ and 11E) SOX9+ cells in relation to distance from injury (− value represents rostral to injury, + value represents caudal to injury). SHAM mice have T7-T9 laminectomies only and are treated with vehicle only. N=6-8. Graphs show mean and SEM. The color of the following symbols # (p<0.1) * (p<0.05) ** (p<0.01) *** (p<0.001) **** (p<0.0001) represents significant difference between that cohort and vehicle (injured). Student's T-test.

FIGS. 12A-12E: Liver fibrosis mitigated with 5c(i) treatment. 8-week-old male mice with a severe T8 contusion/compression SCI were injected IP daily with 5c(i) for 7 weeks (5 and 25 mg/kg/day). 12A) 7 weeks post injury, livers were sectioned and stained with Masson's trichrome to quantify collagen deposition. 7 weeks post injury, heparinized plasma was collected and 12B) alanine aminotransferase (ALT), 12C) alkaline phosphatase (ALP), 12D) aspartate aminotransferase (AST), and 12E) blood urea nitrogen (BUN) levels were assessed. SHAM mice have T7-T9 laminectomies only and are treated with vehicle only. N=6-8. Graphs show mean and SEM. # (p<0.1) * (p<0.05) ** (p<0.01) *** (p<0.001) **** (p<0.0001). Student's T-test.

### DETAILED DESCRIPTION

### I. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “subject,” as used herein, generally refers to an individual having a medical condition treatable with 5c(i) and/or a functional derivative or that is suspected of having a medical condition treatable with 5c(i) and/or a functional derivative. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as a neurological disorder. The subject may be undergoing treatment or having undergone treatment. The subject may be asymptomatic. The subject may be healthy individuals but that are desirous of prevention of a neurological disorder. The term “individual” may be used interchangeably, in at least some cases. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants and includes in utero individuals. It is not intended that the term connote a need for medical treatment, therefore, an individual may voluntarily or involuntarily be part of experimentation whether clinical or in support of basic science studies.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of one or more symptoms of the disease or condition, or the delaying of the progression of the disease or condition. “Treatment” does not necessarily indicate complete eradication or cure of the disease or condition, or associated symptoms thereof. Treating may mean alleviation of at least one symptom of the disease or condition.

As used herein, the term “functional derivative” refers to a compound comprising a peptide linked to a dye with structural similarity to 5c(i) and that retains at least 10, 20, 30, 40, 50, 60, 70, 80, or 90% or greater of the activity of 5c(i). More than one activity for the functional derivative may be measured to determine functionality. In specific embodiments, the activity being measured is modulation of TrkB and/or TrkC. In some embodiments, the activity being measured is activating TrkB and/or TrkC. In certain embodiments, the activity being measured is binding TrkB and/or TrkC. In some embodiments, the activity being measured is modulation of any pathway upstream or downstream of TrkB and/or TrkC. In some embodiments, the activity being measured may include interaction with GSK3beta, Complement competent 3 (C3), and/or Synapsin I, and in specific cases such interaction produces alternative methods of action. In some embodiments, the activity being measured concerns a pathway relating to or connected by GSK3beta, C3, and/or Synapsin I. In specific embodiments, the activity being measured comprises the ability to increase wound healing; the ability to modify astrogenesis; the ability to increase the number of pro-regenerative astrocytes; the ability to increase neurite regeneration, sprouting, and/or sparing; the ability to increase axon regeneration, sprouting, and/or sparing; the ability to increase cell survival and/or neuroprotection; the ability to increase synaptogenesis; the ability to reduce levels of CD68 (e.g., the ability to reduce levels of CD68 systemically and/or locally in any region, especially central and peripheral nervous systems and/or key organs (heart, spleen, liver, lungs, and/or GI tract); the ability to reduce inflammation; the ability to target stem cell differentiation and/or proliferation in one or more of the following locations: the peripheral nervous system (PNS), Schwann cells located in the PNS, glial cells (astrocytes, oligodendrocytes, microglia, Shawn cells, ependymal cells or a combination thereof), hepatic stem/progenitor cells, and/or intestinal cells.

The term “secondary pathology” or the like as used herein refers to any pathology associated with or resulting from a primary pathology (disease/disorder) that is not the primary phenotype. In specific embodiments, it comprises one or more diseases that stem from a change in one organ or tissue as a result of a disease in another organ or tissue. In specific embodiments, it refers to a disease or disorder or symptom that is the direct or indirect result of a neurological disorder and/or injury to the central and/or peripheral nervous system.

The term “alkyl,” by itself or as part of another substituent, means, unless otherwise stated, a linear (i.e. unbranched) or branched carbon chain, which may be fully saturated, mono- or polyunsaturated. An unsaturated alkyl group is one having one or more double bonds or triple bonds. Saturated alkyl groups include those having one or more carbon-carbon double bonds (alkenyl, also olefinic) and those having one or more carbon-carbon triple bonds (alkynyl). The groups, —CH₃ (Me), —CH₂CH₃ (Et), —CH₂CH₂CH₃ (n-Pr), —CH(CH₃)₂ (iso-Pr), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), are all non-limiting examples of alkyl groups. The alkyl groups employed herein can be substituted or unsubstituted.

The term “aryl” means a polyunsaturated, aromatic, hydrocarbon substituent. Aryl groups can be monocyclic or polycyclic (e.g., 2 to 3 rings that are fused together or linked covalently). The term “heteroaryl” refers to an aryl group that contains one to four heteroatoms selected from N, O, and S. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. The aryl groups employed herein can be substituted or unsubstituted.

The term “acyl” is defined as a carbonyl radical attached to an alkyl, alkenyl, alkynyl, cycloalkyl, heterocycyl, aryl or heteroarvi group, examples including, without limitation, such radicals as acetyl and benzoyl.

The term “heterocyclyl” as used herein represents a saturated 3 to 8 membered ring containing 1 to 3 heteroatoms selected from nitrogen, oxygen and sulfur. Representative examples are indolyl, pyrrolidyl, piperidyl, piperazinyl, morpholinyl, thiomorpholinyl, aziridinyl, tetrahydrofuranyl and the like.

Various groups are described herein as substituted or unsubstituted (i.e., optionally substituted). Optionally substituted groups may include one or more substituents independently selected from: halogen, nitro, cyano, hydroxy, amino, mercapto, formyl, carboxy, oxo, carbamoyl, substituted or unsubstituted alkyl, substituted or unsubstituted heteroalkyl, alkoxy, alkylthio, alkylamino, (alkyl)₂amino, alkylsulfinyl, alkylsulfonyl, arylsulfonyl, substituted or unsubstituted cycloalkyl, substituted or unsubstituted heterocyclyl, substituted or unsubstituted aryl, substituted or unsubstituted heteroaryl, and other substituting groups known in the art. In certain aspects the optional substituents may be further substituted with one or more substituents independently selected from: halogen, nitro, cyano, hydroxy, amino, mercapto, formyl, carboxy, carbamoyl, unsubstituted alkyl, unsubstituted heteroalkyl, alkoxy, alkylthio, alkylamino, (alkyl)₂amino, alkylsulfinyl, alkylsulfonyl, arylsulfonyl, unsubstituted cycloalkyl, unsubstituted heterocyclyl, unsubstituted aryl, or unsubstituted heteroaryl. Exemplary optional substituents include, but are not limited to: —OH, oxo (═O), —Cl, —F, Br, C₁₋₃alkyl, phenyl, benzyl, —NH₂, —NH(C₁₋₄alkyl), —N(C₁₋₄alkyl)₂, —NO₂, —S(C₁₋₄alkyl), —SO₂(C₁₋₄alkyl), —CO₂(C₁₋₄alkyl), and —O(C₁₋₄alkyl).

The term “dye” refers to an organic compound organic that absorbs light in the visible spectrum (400-700 nm), has at least one chromophore (color-bearing group), and has a conjugated system of electrons, i.e., a structure with alternating double and single bonds.

### II. Embodiments of Compounds

In certain embodiments, the compound is a modulator of TrkB, TrkC, or both, and in specific embodiments, the compound is an activator of TrkB, TrkC, or both. In specific embodiments, the compound is able to cross the blood-brain barrier and/or blood-spinal cord barrier.

In particular embodiments, compounds of the disclosure for use in the methods comprise a compound that comprises a peptide linked to a dye. In specific embodiments, the compound comprises one or more dye-bound cyclized peptides. The compound may comprise a peptide having a specific amino acid composition. In specific embodiments, the peptide is a 6-mer. In specific embodiments, one or more amino acids are at the N-terminus and/or C-terminus of the 6-mer to link the 6-mer to the dye. In some embodiments, the peptide is not a 6-mer but instead may be a 3-mer, 4-mer, 5-mer, 7-mer, 8-mer, 9-mer, and so forth.

In some embodiments, the peptide of the compound may comprise the 4-mer DIRG. In certain embodiments, a 4-mer, 5-mer, or 6-mer is linked by the N-terminus and the C-terminus to a dye, including a BODIPY® (fluorescent dye) dye, in some cases. In some aspects, a 4-mer, 5-mer, or 6-mer is linked to a dye through an amino acid, or through two amino acids. For example, the peptide C-(DIRG)-C can be linked to a dye through the sulfur atoms on each of the terminal cysteine amino acids. In some cases, the peptide has the structure of C-(AA¹⁻⁴)-C. In some cases, the peptide has the structure DIKG, DMSG, DLRG, INNS, VSKG, TQNS, TGNS, AGGS, DGKQ, DEKQ, DSKK, ENNK, or DAQG. In certain embodiments, the N-terminus and the C-terminus of a 4-mer, 5-mer, or 6-mer are linked to a C₄-C₆ alkyl. In some instances, the N-terminus and the C-terminus of a 4-mer, 5-mer, or 6-mer are linked to a spacer group whose distance between atoms bound to the N-terminus and the C-terminus of the peptide moiety is the same as the distance between BODIPY atoms bound to the N-terminus and the C-terminus of the peptide moiety±1A.

The composition of matter of 5c(i) (as one example), is described in PCT patent application WO2022256394A2, is incorporated by reference herein. In specific cases a DIRG peptide sequence is attached to a fluorescent dye, such as a BODIPY® (fluorescent dye) dye. In some cases, there may be a change of one or more amino acids at D, I, R, or G. In some embodiments, the change is a conservative change of an amino acid. In some instances, an amino acid substitution or replacement is selected such that the replacement amino acid has similar physicochemical properties such as electronegativity, steric size, and lipophilicity as the amino acid being replaced. The substitution of one amino acid of a parent compound with a different amino acid with similar physicochemical properties is performed to provide a resultant compound that elicits similar biological activity as the parent compound. For example, D may be changed to E; I may be changed to G, A, V, or L; R may be changed to H or K, and G may be changed to I, A, V, or L. In cases where the peptide is a 5-mer, 6-mer or longer, the peptide may or may not comprise the sequence DIRG, including as a consecutive sequence of DIRG.

In some embodiments, the dye is a divalent dye. The divalent dye can be represented by formula II below

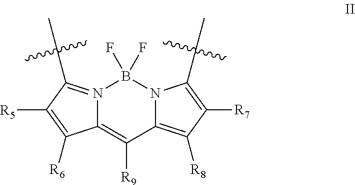

where R₅, R₆, R₇, and R₈ are each independently H, halide, C₁-C₃ alkyl, or SO₃H or a salt thereof; R₅ and R₆, can optionally join to form an aryl ring; R₇ and R₈ can optionally join to form an aryl ring; and R₉ is H, a halide, C₁-C₃ alkyl, aryl, or SO₃H or a salt thereof.

In some embodiments, the dye is rhodamine. When bound to a peptide as disclosed herein, the rhodamine dye can be represented by formula III below

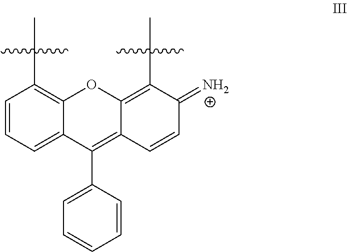 [SMILES: *c1c2oc3c(*)cccc3c(-c3ccccc3)c-2ccc1=[NH2+]]

In further embodiments, the dye is fluorene. When bound to a peptide as disclosed herein, the fluorene dye can be represented by formula IV below

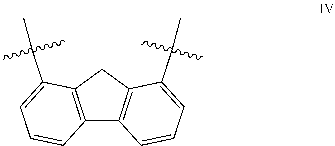 [SMILES: *c1cccc2c1Cc1c(*)cccc1-2]

In some instances, the dye has a fluorescent emission of greater than 490 nm. The fluorescent emission can be measured in water or in an organic solvent such as acetone, pentane, hexane, ethyl acetate, tetrahydrofuran, methylene chloride, chloroform, isopropanol, dimethyl sulfoxide, diethyl ether, carbon tetrachlorid, cyclohexan, butyl acetate, 1,4-dioxan, 1-butanol, and heptane, for example. The dye can have a fluorescent emission of any one of, less than, greater than, between, or any range thereof of 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, or 700 nm. In specific embodiments, the dye is represented below, wherein each * represents a point of covalent attachment to the sulfur atom in a cysteine residue in the sequence:

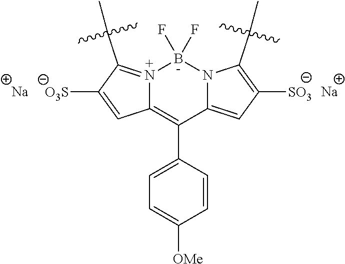

The BODIPY® (fluorescent dye) dye may be synthesized following the general procedure found in the literature (see “Li, L.; Han, J.; Nguyen, B.; Burgess, K., J. Org. Chem. 2008, 73, 1963-1970”). Any BODIPY® (fluorescent dye) dye or a derivative thereof may be utilized (Loudet and Burgess, Chem. Rev. 2007, 107, 11, 4891-4932; or see Poddar and Misra, Coordination Chemistry Reviews. Volume 421, 15 Oct. 2020, 213462)

In some embodiments, the composition has one or more charged groups, although in some embodiments there are no charged groups.

In specific embodiments, the therapeutic composition may comprise a peptide of DIRG. In specific embodiments, the therapeutic composition may comprise a peptide comprising one or more conservative substitutions in the peptide of DIRG. In specific embodiments, the therapeutic composition may comprise a peptide of DIRG linked to a dye. In specific embodiments, the therapeutic composition may comprise a peptide comprising one or more conservative substitutions in the peptide of DIRG, wherein the peptide is linked to a dye. Any peptide of the disclosure may be linked to the dye through 1, 2, or more amino acids. Any peptide of the disclosure may be linked to the dye at the N-terminus and C-terminus. Any peptide of the disclosure may be linked to the dye through one or more amino acid side chains. Any peptide of the disclosure may be linked to the dye at the N-terminus and C-terminus and each linkage is the same amino acid. Any peptide of the disclosure may be linked to the dye at the N-terminus and C-terminus and each linkage has a different amino acid. In specific cases, any peptide of the disclosure may be linked to the dye at the N-terminus and C-terminus and each linkage may or may not be a cysteine.

### III. Embodiments of Uses of the Compounds

In particular embodiments, the disclosure encompasses methods of use for a compound that comprises one or more dye-bound cyclized peptides for one or more certain medical conditions. In specific embodiments, the disclosure encompasses methods of use for one or more dye-bound cyclized peptides and/or a functional derivative thereof for neurological indications and all other pathologies associated with those neurological indications.

In particular embodiments, there are methods of improving locomotor function, improving motility, improving long-term memory, improving cognition, reducing fibrosis (including liver fibrosis), reducing inflammation, treating cancer, and so forth, upon treatment with 5c(i) and/or a functional derivative thereof for an individual in need thereof.

In particular embodiments, there are methods of treating neuroinflammation, including neuroinflammation associated with various neurological disorders, neurodegenerative diseases, traumatic brain injury, bacterial, viral, and fungal meningitis, and stroke, as examples. In specific embodiments, an individual in need thereof is an individual that has or is at risk for having a neurological disorder, neurodegenerative disease, traumatic brain injury, bacterial meningitis, viral meningitis, fungal meningitis, or stroke.

Examples of neurological disorders (e.g., diseases of the brain, spine, and the nerves that connect them) include at least Alzheimer's Disease, multiple sclerosis, frontotemporal dementia, psychiatric disorders (such as Anxiety disorders, Schizophrenia, Behavioural and emotional disorders, Bipolar affective disorder, Depression, Dissociation and dissociative disorders, Eating disorders, Obsessive compulsive disorder, Paranoia), Amyotrophic Lateral Sclerosis (ALS), ataxia, Bell's Palsy, brain tumor, cerebral aneurysm, encephalitis, epilepsy, seizures, Guillain-Barre Syndrome, dementia, migraine, or Parkinson's Disease. An individual with any neurological disorder may be subject to methods and compositions of the disclosure. An individual with symptoms such as headache, sciuri, loss of feeling or tingling, weakness or loss of muscle strength, loss of sight or double vision, memory loss, impaired mental ability, lack of coordination, and/or stroke may be subject to methods and compositions of the disclosure. An individual may be subject to methods and compositions of the disorder who is at risk for a neurological disorder, such as having an associated genetic mutation and/or a family and/or personal history.

Examples of neurodegenerative diseases (e.g., when cells of the central nervous system stop working or die) include at least Alzheimer's disease, Amyotrophic lateral sclerosis, Friedreich ataxia, Huntington's disease, Lewy body disease, Parkinson's disease, or Spinal muscular atrophy. In particular embodiments, the individual with the neurodegenerative disorder gets worse over time. The neurodegenerative disease may be caused by any reason, such as an associated genetic mutation, from a tumor, from a stroke, be an alcoholic, exposure to toxins (mercury, aluminum, copper, lead, manganese, β-N-methylamino-L-alanine), exposure to chemicals (pesticides, metal-based nanoparticles), exposure to certain viruses (Herpes simplex virus, varicella zoster virus, some strains of influenza virus, and viruses that cause encephalitis or meningitis), or have an unknown cause. An individual may have one or more of the following symptoms prior to onset of treatment with the methods and compositions of the disclosure: a loss of inhibition, anxiety, agitation, apathy, difficulty with movement, forgetfulness, memory loss, and mood changes. In particular embodiments, the methods and compositions prevent the onset of one or more symptoms, delay the onset of one or more symptoms, or reduce the severity of one or more symptoms.

For some medical conditions, the condition may be considered both a neurological disorder and a neurodegenerative disease.

An individual may have Traumatic brain injury (TBI) including brain dysfunction that may be caused by an outside force, such as a violent blow to the head. It may occur as a result of a severe sports injury, a vehicle accident, etc. In some embodiments, it is followed by immediate or delayed symptoms, such as confusion, blurry vision, concentration difficulty, headache, convulsions or seizures, blurred or double vision, unequal eye pupil size or dilation, etc. The TBI may be a penetrating TBI or a non-penetrating TBI.

In particular embodiments, there are methods of improving locomotor function and/or long-term memory, including after CNS trauma, such as for an individual with spinal cord injury. In some embodiments, there are methods of treating one or more neurological disorders, including at least Parkinson's and Alzheimer's disease, stroke, dementia, neurotrauma, amyotrophic lateral sclerosis, and multiple sclerosis, as examples. In some embodiments, there are methods of treating a neurological indication, such as one relating to cognition and mobility, including age-associated neurological conditions, such as neurodegenerative diseases and neurotrauma.

In particular embodiments, there are methods of treating one or more secondary pathologies associated with a primary pathology (e.g., neurological indications). In specific embodiments, there are methods for individuals after spinal cord injury-induced paralysis of reducing liver fibrosis, reducing inflammation, reducing markers for inflammation (e.g., C-reactive protein (CRP), erythrocyte sedimentation rate (ESR), and procalcitonin (PCT), serum amyloid A, cytokines, alpha-1-acid glycoprotein, plasma viscosity, ceruloplasmin, hepcidin, and/or haptoglobin), cancer, and so forth.

Although in some embodiments, the method is for treating a primary pathology and/or a second pathology in an individual, in certain embodiments any medical condition referred to herein as a second pathology is instead treated as the primary pathology itself.

In particular embodiments, there are methods of treating any pathological conditions resulting from a consequence of spinal cord injury or any other neurotraumatic injury, including at least the following: cardiac fibrosis, liver fibrosis, damage to any of the other organs (fibrosis, necrosis, dysfunction, and inflammation to those organs), liver, kidney, and colon diseases, any cardiac and metabolic pathologies (including diabetes, cardiometabolic syndrome, and cardiovascular disease), muscle-associated diseases, respiratory diseases, and cancer.

In some embodiments, the methods include treatment for any neurotrauma (head or spine injury caused by a sudden injury and/or peripheral neuropathy/neurotrauma and neuropathies). In specific embodiments, neurotrauma includes at least concussions, TBI, skull fractures, spinal column fractures, and spinal cord injuries. In certain embodiments, the individual has a head trauma, such as a concussion, mild TBI, moderate TBI, severe TBI (such as involving a coma), penetrating brain injury (such as a gunshot or stab wound), depressed skull fracture, craniofacial trauma, epidural hematoma, acute subdural hematoma, subacute subdural hematoma, chronic subdural hematoma, traumatic subarachnoid hemorrhage, traumatic intracerebral hemorrhage/brain contusion, traumatic cerebrospinal fluid leak, traumatic pseudoaneurysm, and blunt cerebrovascular injury. In certain embodiments, the individual has spine trauma, such as craniometrical spine injury, cervical spine injury, thoracic spine injury, lumbosacral spine injury, atlanto-occipital dissociation, Jefferson (C1) fracture, Hangman's (C2) fracture, odontoid (dens) fracture, traumatic central cord syndrome, perched/jumped facets, compression (anterior wedge) fracture, burst fracture, chance fracture, fracture-dislocation, traumatic pars defect (spondylolysis), traumatic spondylolisthesis, traumatic intervertebral disc herniation, complex sacropelvic injury, hyperflexion injury, hyperextension injury, compression injury, distraction injury, and spinal ligamentous injury.

In some embodiments, the disease being treated is cancer, whether or not the individual has another medical condition, such as a neurological disorder and/or injury to the central and/or peripheral nervous system. Cancers for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

Embodiments of the disclosure include methods of treating neurotrauma in an individual in need thereof, comprising administering to the individual one or more compounds of the present disclosure.

Embodiments of the disclosure include methods of treating neurodegenerative disease in an individual in need thereof, comprising administering to the individual one or more compounds of the present disclosure.

Embodiments of the disclosure include methods of improving neuron survival in an individual in need thereof, comprising administering to the individual one or more compounds of the present disclosure.

Embodiments of the disclosure include methods of improving neuritogenesis in an individual in need thereof, comprising administering to the individual one or more compounds of the present disclosure.

Embodiments of the disclosure include methods of improving neuronal plasticity in an individual in need thereof, comprising administering to the individual one or more compounds of the present disclosure.

Embodiments of the disclosure include methods of regenerating neurites in an individual in need thereof, comprising administering to the individual one or marc compounds of the present disclosure. Embodiments of the disclosure include methods of enhancing neuron survival in an individual in need thereof, comprising administering to the individual one or more compounds of the present disclosure. Embodiments of the disclosure include methods of reducing inflammation of astrocytes (in specific embodiments, as measured by the amount of Synapsin I. C3, and/or GSK-3β) in an individual in need thereof, comprising administering to the individual one or more compounds of the present disclosure. Embodiments of the disclosure include methods of enhancing neuron health (in specific embodiments, as reflected by the amount of neurite outgrowth and/or the number of surviving neurons) in an individual in need thereof, comprising administering to the individual one or more compounds of the present disclosure.

Embodiments of the disclosure include methods of reducing an injury size in an individual in need thereof, comprising administering to the individual one or more compounds of the present disclosure. The injury may be in any part of the individual, but in specific embodiments the injury is in the brain; kidney; lung; colon; eye; arm; leg; throat; stomach; breast; abdominal cavity; adrenal gland; aorta; bone; ear, eye; heart; kidney; large intestine; lung; nose; ovary; pancreas; pituitary gland; small intestine; spinal cord; spleen; stomach; testis; thymus; thyroid gland; tooth; uterus; vertebral column, etc. The injury may be in any tissue in the body, including connective tissue, epithelial tissue, muscle tissue, nervous tissue, or a combination thereof.

Embodiments of the disclosure include methods of reversing paralysis in an individual in need thereof, comprising administering to the individual one or more compounds of the present disclosure. The paralysis that is reversed may be of any kind, including Monoplegia, Hemiplegia, Paraplegia, or Quadriplegia. The paralysis being reversed may be partial or complete. The paralysis may or may not have been caused by a stroke, spinal cord injury, nerve disorder (e.g., multiple sclerosis), Bell's palsy, and so forth.

Embodiments of the disclosure include methods of enhancing memory in an individual in need thereof, comprising administering to the individual one or more compounds of the present disclosure. The type of memory may be of any kind, including episodic memory, semantic memory, procedural memory, short-term memory, working memory, sensory memory, and long-term memory. In specific embodiments, the compound promotes long-term memory and/or short-term memory.

Embodiments of the disclosure include methods of causing or enhancing regeneration of any cell type, including in a non-cancerous matter, including regenerating liver cells and neural cells (e.g, neurons, Schwann cells, astrocytes, and/or microglia and stem cells from which these cells are derived) that is healthy to have regenerated in a non-cancerous, non-inflammatory, and/or non-diseased manner in an individual in need thereof, comprising administering to the individual one or more compounds of the present disclosure. Embodiments of the disclosure methods of causing or enhancing regeneration of axons and/or glial processes manner in an individual in need thereof, comprising administering to the individual one or more compounds of the present disclosure.

Embodiments of the disclosure include methods of reducing inflammation in an individual in need thereof, comprising administering to the individual one or more compounds of the present disclosure. In specific embodiments, the inflammation may be acute or chronic. In specific embodiments, reduction of inflammation encompasses reduction of disease-inducing inflammatory pathways (e.g., pathways that include NF-κB, MAPK, JAK-STAT, IL-1, IL-6, TNF-α, lymphotoxin, IFN-γ, IL-17A, IL-10, IL-1b, any MMP (including MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, MMP28), and/or IL-13), either systemically or locally, through any mechanism, either neural or otherwise.

Embodiments of the disclosure include methods of mitigating fibrosis in an individual in need thereof, comprising administering to the individual one or more compounds of the present disclosure. In certain embodiments, the fibrosis may be of any kind. The fibrosis may be in the lung, liver, heart, lymph nodes, retroperitoneum, bone marrow, skin, connective tissue, or a combination thereof, in specific embodiments. In some embodiments, the fibrosis is not in the lung, liver, gastrointestinal tract, peripheral nervous system, central nervous system, heart, lymph nodes, retroperitoneum, bone marrow, skin, connective tissue, or a combination thereof.

Embodiments of the disclosure include methods of enhancing cognition in an individual in need thereof, comprising administering to the individual one or more compounds of the present disclosure.

Embodiments of the disclosure include methods of enhancing neurite growth in an individual in need thereof, comprising administering to the individual one or more compounds of the present disclosure. In specific embodiments, the neurite that is a projection from the cell body of a neuron may be either an axon or a dendrite, or both may occur.

Embodiments of the disclosure include methods of providing neuroprotection against oxidation and ischemia in an individual in need thereof, comprising administering to the individual one or more compounds of the present disclosure.

Embodiments of the disclosure include methods of modulating astrocytes to increase Synapsin I expression and/or reduce C3 and GSK-3β expression in an individual in need thereof, comprising administering to the individual one or more compounds of the present disclosure. In specific embodiments, the individual has spinal cord injury, Alzheimer's Disease, or any medical condition in which altered levels of Synapsin I, one or more neurotrophic factors (e.g., BDNF, NT-3, and/or NT-4), C3 and/or GSK-3β is deleterious.

Embodiments of the disclosure include methods of promotion of astrocyte migration and/or proliferation in an individual in need thereof, comprising administering to the individual one or more compounds of the present disclosure.

Embodiments of the disclosure include methods of enhancement of astrocyte polarization in an individual in need thereof, comprising administering to the individual one or more compounds of the present disclosure.

Embodiments of the disclosure include methods of penetrating of the blood-brain barrier (BBB) and blood-spinal cord barrier (BSCB) with one or more activators of TrkB/TrkC in an individual in need thereof, comprising administering to the individual one or more compounds of the present disclosure.

Embodiments of the disclosure include methods of improving functional locomotor recovery in an individual in need thereof, comprising administering to the individual one or more compounds of the present disclosure.

Embodiments of the disclosure include methods of improving cognition after SCI in an individual in need thereof, comprising administering to the individual one or more compounds of the present disclosure.

Embodiments of the disclosure include methods of cognitive dysfunction in an individual in need thereof, comprising administering to the individual one or more compounds of the present disclosure.

Embodiments of the disclosure include methods of improving memory function in individuals, such as with cognitive dysfunction in an individual in need thereof, comprising administering to the individual one or more compounds of the present disclosure.

Embodiments of the disclosure include methods of improving memory in an individual in need thereof, such as with a disease that reduces memory function (e.g., Alzheimer's disease, Dementia with Lewy Bodies, Frontotemporal Dementia, HIV Dementia, Mild Cognitive Impairment, Dementia related to Normal Pressure Hydrocephalus, Vascular Dementia, Hyperthyroidism, stroke, head injury, spinal injury, Alcoholism, Sleep apnea, Nutritional deficiency, Parkinson's disease, Depression, cancer, chemo brain, neurosyphilis, Creutzfeldt-Jakob disease, Huntington's disease, and/or infection), comprising administering to the individual one or more compounds of the present disclosure.

Embodiments of the disclosure include methods of enhancing axon regeneration, axon survival, and/or axon sprouting in an individual in need thereof, comprising administering to the individual one or more compounds of the present disclosure.

Embodiments of the disclosure include methods of mitigating one or more liver pathologies in an individual in need thereof, comprising administering to the individual one or more compounds of the present disclosure. In specific embodiments, the liver pathology is nonalcoholic fatty liver disease, and in some cases the nonalcoholic fatty liver disease is in an individual with SCI.

Embodiments of the disclosure include methods of mitigating any disease that directly or indirectly affects liver function in an individual in need thereof, comprising administering to the individual one or more compounds of the present disclosure.

Individuals being treated by methods of the disclosure may be of any sex (including those with Sex Chromosome Aneuploidy) and of any age and of any race. In some embodiments, the individual may be an individual that is not at risk for a neurological disorder, whereas in other embodiments the individual is an individual that is at risk for a neurological disorder. An individual at risk may be an individual that has advanced age, a genetic disorder, congenital abnormality or disorder, infection, lifestyle or environmental health problems including malnutrition, and brain injury, spinal cord injury or nerve injury, in some embodiments. In specific cases, advanced age is an individual at least 60, 65, 70, 75, 80, 85, 90, 95, or 100 or more years old. In some cases, the individual is being born, is a newborn, is an infant (less than one year old), or is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 or more years old.

In some embodiments, any compound encompassed herein (comprising one or more dyes) may be used for tracking the compound itself for any purpose. Examples include to determine mechanism of action of one or more of the compounds, to determine pharmacokinetic properties of one or more of the compounds, and/or to determine toxicity of one or more of the compounds. In some embodiments one or more of the compounds are utilized as a tracking system to localize targets of the compound, such as a result of binding (indirectly or directly) of the compound to one or more targets. In specific embodiments, the molecule to which the compound binds is one or more of a protein, nucleic acid, carbohydrate, small molecule, biologic, metabolite, lipid or a combination thereof. The compound and the molecule to which the compound binds may or may not be in a complex with other components.

In certain embodiments, there is a method of diagnosis and/or prognosis in which an effective amount the compound is delivered to an individual in need of diagnosis and/or prognosis, and the location (and in some cases movement) of the compound is visualized and such tracking provides diagnostic and/or prognostic for the individual. The individual may be known to have a medical condition, suspected of having a medical condition, or at risk of having a medical condition (for example, a risk greater than the general population). Any individual at risk for having a medical condition may have a personal or family history, may have one or more behaviors and/or environments that makes them prone to the medical condition, may have one or more genetic markers that makes them prone to the medical condition, a combination thereof, and so forth.

In certain embodiments, the method comprises monitoring efficacy of a therapy for the individual. The individual may be provided an effective amount of the compound prior to a therapy that is not the compound itself, and at certain time points of duration following such an initial administration, the individual is provided the compound to determine if the therapy is being effective. Efficacy of the therapy may manifest as location of the compound, the intensity of the signal from the compound, or a combination thereof. The therapy may or may not be continued based on such a determination.

In some embodiments, the individual is provided an effective amount of the compound wherein the compound itself is the therapy. At a subsequent time point, the individual is subjected to visualization to identify the location of the compound, the intensity of the signal from the compound, or a combination thereof. Between the initial administration of the compound and a subsequent one or more time points, the individual may be occasionally or continuously monitored for the location of the compound, the intensity of the signal from the compound, or a combination thereof.

### IV. Administration of Therapeutic Compositions

The therapies provided herein may comprise administration of one or a combination of 5c(i) and/or functional derivatives thereof. Embodiments of the disclosure relate to compositions and methods comprising therapeutic compositions. When multiple therapies are provided, the different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

In some embodiments, the therapy is administered at a dose of between 1 mg/kg and 5000 mg/kg. In some embodiments, the therapy is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg.

In some embodiments, a single dose of the 5c(i) and/or functional derivative therapy is administered. In some embodiments, multiple doses of the 5c(i) and/or functional derivative therapy are administered. In some embodiments, the 5c(i) and/or functional derivative therapy is administered at a dose of between 1 mg/kg and 100 mg/kg. In some embodiments, the 5c(i) and/or functional derivative is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The compounds disclosed herein may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S- or the R-configuration, as defined by the IUPAC 1974 Recommendations. Compounds may be of the D- or L-form, for example. It is well known in the art how to prepare and isolate such optically active forms. For example, mixtures of stereoisomers may be separated by standard techniques including, but not limited to, resolution of racemic form, normal, reverse-phase, and chiral chromatography, preferential salt formation, recrystallization, and the like, or by chiral synthesis either from chiral starting materials or by deliberate synthesis of target chiral centers.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions may be via any suitable route. This includes, but is not limited to oral, intravenous administration, orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions may be administered in a manner compatible with the dosage formulation and in such amount as is therapeutically or prophylactically effective. The formulations are easily administered in a variety of dosage forms, such as the type of injectable solutions described above.

In certain aspects, the compositions or agents for use in the methods, such as 5c(i) and/or functional derivatives thereof, are suitably contained in a pharmaceutically acceptable carrier. The carrier may be non-toxic, biocompatible and may be selected so as not to detrimentally affect the biological activity of the agent. The agents in some aspects of the disclosure may be formulated into preparations for local delivery (i.e., to a specific location of the body, such as nervous or other tissue) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol may be between about 0.01 ml and 0.5 ml, for example.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Examples of Methods and Materials

Embodiments of the present disclosure are encompassed in the present example. In specific embodiments, the following methods were utilized, although the skilled artisan recognizes that modifications may be made to the methods, in certain embodiments.

### Examples of Methods

### Animals

All studies herein use wild-type C57Bl/6 mice or other variations, including, C57Bl/6N, C57Bl/6NJ, C57Bl/6J, and B6NTac mice. All procedures were conducted according to the protocol approved by the Institutional Review Board/Animal Ethics Committee of Texas A&M University.

### Cell Culture

For all cell culture experiments, primary neural cells were extracted from the cortices of postnatal mice. The extraction, purification, and separation of neurons and glia was conducted as previously before⁵⁸ covered under patent application WO2022266431, incorporated by reference herein in its entirety. Briefly, after euthanization of mice, the brains were microdissected and placed in papain filled GENTLEMACS™ (tissue dissector) C Tubes followed by cell dissociation using the GENTLEMACS™ (tissue dissector) Octo Dissociator with Heaters with heating cuffs using the GENTLEMACS™ (tissue dissector) Program 37C_ABDK_01 protocol. After protocol completion, the contents were strained through a 70 μm cell strainer, debris and red blood cells were removed, and the remaining solution was labeled with glia specific antibodies. The MACS magnetic sorting system was utilized to separate the labeled glia from the remaining neuron enriched cell population.

For neuronal cultures, the neuron enriched fraction was added onto plastic bottom (Greiner-Bio, 781091) plates and incubated inside a 5% C02 incubator at 37° C. for 2 days. Pharmaceutical agents were added at the time of cell plating. For astrocyte cultures, the glia fraction was added onto plastic bottom (Greiner-Bio, 781091) plates and incubated inside a 5% C02 incubator at 37° C. for 5 days for 100% confluency to be reached. Then, the astrocytes were scratched to form the scratch assay model and the pharmaceutical agents were added at the time of scratching. 10,000 cells were plated per well with 0.056 cm² growth area for both the neuronal and glia cultures.

### Immunocytochemistry

Cell cultures were fixed with 4% paraformaldehyde (PFA, 15 min) after the completion of the respective in vitro experiment. After fixation, immunocytochemistry was conducted by first washing the cells with phosphate-buffered saline (PBS) 3 times, then incubating in 5% normal horse serum for 60 min to block nonspecific binding (VWR® (laboratory and science products), 102643-676). Afterwards for neuronal cultures, the neurons were incubated in 1:500 TUBB3 (BIOLEGEND® (scientific reagents), 801202) for 24 hours followed by being washed 3 times with PBS and incubated in 1:500 Alexa Flour 488 (THERMO FISHER SCIENTIFIC® (laboratory reagents), A32723) and 1:10,000 DAPI (VWR, 95059-474) for 60 min, all conducted at room temperature. This allows for the analysis of morphological features as previously conducted⁵⁸. Astrocyte cultures for scratch assays were incubated in 1:500 DYLIGHT™ (scientific reagents) 554 and 1:10,000 DAPI (VWR® (laboratory and science products), 95059-474) for 60 min at room temperature. This allows for the analysis of the scar size in culture. After the completion of immunocytochemistry, the cells were preserved in FLUOROMOUNT-G® (slide mounting media) Mounting Medium (THERMO FISHER SCIENTIFIC® (laboratory reagents), 00-4958-02) until imaging. All experimentation after incubation in secondary antibody was performed in the absence of light.

### Protein Kinetics

Transfected cell lines that are Trk positive cells (TrkA-HeLa, TrkB-HEK293, and TrkC-NIH/3T3) and non-transfected cell lines that are Trk negative cells (HeLa, HEK293, and NIH/3T3) were seeded at 2,000 cells/well in a 96-well plate (0.32 cm² growth area) and incubated inside a 5% CO₂ incubator at 37° C. for 24 hours. The cells were treated with different concentrations of pharmaceutical agents in serum-free media with and without 0.2 nM of NGF (TrkA-HeLa and HeLa), 0.6 nM of BDNF (TrkB-HEK293 and HEK293), and 0.2 nM of NT-3 (TrkC-NIH/3T3 and NIH/3T3) for 150 min. Afterwards, the cells were washed with 1% sodium dodecyl sulfate PBS once to remove unbound compounds. Cell-associated fluorescence was determined by measuring the emission of the resulting solution upon λₑₓ (540/25 nm) and λₑₘ (620/40 nm) using AGILENT® BioTek Synergy H4 Hybrid Microplate Reader. The dissociation constant (Kd) was calculated using binding saturation (One site—Specific binding).

### Preclinical SCI Model

Male mice at 8 weeks of age underwent surgery to sustain an SCI. The spinalis thoracis muscle was lacerated to perform a laminectomy at T7-T9 to expose the spinal cord while keeping the dura intact. The mice were then clamped using 2 pairs of Adson forceps before using the Infinite Horizon Impactor at 50 kdyn of force (the contusion) with a 2 second dwell time (the compression) to induce a severe T8 contusion and compression. Self-degrading Catgut sutures were utilized for the closure of the spinalis thoracis muscle before the skin was glued shut with 3M VETBOND™ (n-butyl cyanoacrylate adhesive veterinary tissue adhesive) Tissue Adhesive. All procedures and instruments used for surgery were sterilized prior to surgery to prevent infections. After surgery, mice recovered in a warmed cage and then moved to a cage set at room temperature provided with food and water ad libitum. All surgeries were performed in the morning (8 am-12 pm) to limit potential circadian clock impact. Behavioral assays were performed at the same time in the morning for all the cohorts for the same reason. For the administration of pharmaceutical agents, the vehicle was 0.6 mL/kg of 100% dimethyl sulfoxide (DMSO). 5c(i) (at 5 and 25 mg/kg/day) diluted in vehicle or vehicle alone was administered once daily through intraperitoneal (IP) injections at a volume of 0.6 mL/kg/day. Bladders were expressed manually twice daily (morning and evenings) to minimize risk of bladder ruptures.

### Locomotor Tests

Open Field Basso Mouse Scale (BMS): Mice were placed in an open field for 5 minutes to allow mice to freely roam to visually evaluate (by 2 individuals) the following parameters: ankle movements, stepping pattern, coordination, paw placement, trunk instability, and tail position. The mice were scored according to the BMS scoring system⁵⁹ with a minimum score of 0 (no movement) to a maximum score of 9 (normal locomotion) followed by the BMS Sub score (assessing stepping patterns, tail movement, trunk posture, paw position, and coordination)^{12,60}. Rotarod: Mice were placed on a rod rotating at increasing speeds from 5 to 45 rotations per minute (rpm) in a 3-minute interval with constant acceleration to assess strength, endurance, and motor coordination^{12,60}. Mice were first acclimated to the rotarod with 5 habituation sessions the week before injury. Baseline testing was conducted 1 day before injury.

### Cognitive Tests

Before the start of the study, uninjured mice were placed in the room all cognitive tests are conducted in for 1 hour. Afterwards, mice were placed in an empty open field white box for 5 min to habituate to the box and recorded for baseline measurements and identification of abnormalities. 7 weeks after SCI, all post injury cognitive testing was conducted. Novel Object Recognition (NOR): Mice were placed in the empty open field white box for 5 min and recorded. 2 hours later, mice were placed in the same box with 2 identical objects, each with an approximate volume of 100 cm³, for 10 min. 24 hours after habituation with 2 identical objects, the mice were placed with 1 object identical to the previous days testing (familiar object), and another object (novel object), different in color and shape, but identical in size, for 10 min. Behavioral patterns with respect to the novel and familiar objects was recorded and analyzed. Y-Maze: Forced alteration Y-Maze was conducted by first habituating mice to the Y-Maze with the novel arm blocked with a cardboard box for 8 min. 3 hours later, mice were placed inside the Y-Maze with access to all arms, including the novel arm. Behavioral patterns with respect to the arms the mice visited were recorded and analyzed. Observers were blinded to the groups. All cognitive tests were conducted under red lighting only.

### Histological Analyses

Mice were sacrificed 7 weeks after injury by a lethal dose of Fatal plus (pentobarbital), then, transcardial perfusion was performed with PBS-Heparin (10,000 unit/L, 20 mL, 5 mL/min) followed by a 4% paraformaldehyde fixative solution (30 mL/mouse, at 5 mL/min). Right after perfusion, tissues were immersed in 4% PFA for 24 hours, transferred to 15% sucrose for 24 hours, then transferred to 30% sucrose for 24 hours before being embedded. Subsequently, the spinal cords (at injury site±1 mm) were cut into 25 μm-thick transverse sections using a freezing microtome (THERMO FISHER SCIENTIFIC® (laboratory reagents), Microm HM550). For immunohistochemistry (IHC), the spinal cord tissue sections were placed in a blocking solution (5% BSA, 0.4% Triton X-100, PBS) for 1 hour at room temperature. This was followed by overnight incubation with primary antibodies at room temperature. The tissue sections were subsequently washed 3 times with 0.4% Triton X-100-PBS, followed by 1 hour incubation at room temperature with the appropriate secondary antibodies (1:500, INVITROGEN™ (laboratory reagents) ALEXA FLUOR® (fluorescent dyes) Plus series). The primary antibodies included biotinylated NeuN (EMD MILLIPORE® (pharmaceuticals and chemicals), MAB377B, 1:500), rat GFAP (FISHER SCIENTIFIC® (laboratory reagents), 130300, 1:500), rabbit Iba1 (FUJIFILM® (camera components) Wako Chemicals, 019-19741, 1:500), rat CD68 (BIO-RAD®, (electrophoresis and chromatography materials) MCA1957, 1:500), goat SOX9 (R&D SYSTEMS® (biological reagents), AF3075, 1:500), and rabbit anti-5-HT (ImmunoStar, 20080, 1:500). Sections were stained with DAPI (1:5000, Millipore Sigma) to visualize the nucleus. The images were acquired using VS120 Virtual Slide Microscope (Olympus) and Axio Observer 7 (ZEISS® (optical products)) and analyzed with QuPath 0.4.3. The contour (polygonal) tool in the Zen 3.2 (Carl Zeiss AG, Jena, Germany) software was used to trace and measure lesion and cavity size. The lesion size was measured by tracing the glial scar border labeled with GFAP surrounding a DAPI⁺ inner region. Cavity size was measured by measuring the region within the spinal cord without any visible nuclei yet surrounded by GFAP labeled glial scar. Three spinal cord sections per animal were imaged and analyzed. The 3 sections included the section containing the largest lesion size along with sections 300 μm rostral and caudal. Images were quantified using QuPath software. Eight boxes with a width of 200 μm were drawn both rostral and caudal of the injury epicenter. A contour polygon was made around the spinal cord in each of the sixteen boxes. Fluorescent intensity and cell counting was conducted using automated QuPath software. Histological analysis on liver tissue was conducted by sectioning the left lobe of the liver into 8 μm-thick transverse sections. The liver sections were stained with Masson's trichrome staining kit (SIGMA-ALDRICH® (chemical supplies), HT15-1KT) according to the manufacturer's instructions to examine perivascular accumulation of collagen in the liver. Collagen accumulation was quantified using automated QuPath software to determine the extent of fibrosis.

### Blood Cytokine Analysis

MESO SCALE DISCOVERY® (biological assays) (MSD) U-PLEX® (biological assay) Assay Platform was utilized for the cytokine analysis herein. First, whole blood was collected in heparinized tubes immediately before mice sustained an SCI (baseline) and immediately before mice were perfused (final). The tubes were spun at 14,000×g for 14 min before the plasma from the tubes were collected. The plasma was then used to assess changes in blood cytokine levels. For the cytokine measurements, individual U-PLEX® (biological assay) linker-coupled antibodies and coating solution were prepared and used to coat proprietary MSD 96-well plates which were then incubated for 24 hours at 4° C. and washed with PBS. Next, an 8-point calibration curve with 4-fold serial dilution steps was created; 50 μL of it was loaded onto the MSD plate, along with 25 μL of diluent for the wells reserved for the mouse samples. 25 μL of each of the mouse samples were loaded into the wells creating a 2-fold dilution of the mouse samples in diluent. The MSD plate was incubated at room temperature for 2 hours before the MSD plate was washed with PBS. Detection antibody solution was created and loaded onto the plate, followed by a 1 hour incubation at room temperature and washing with PBS. Finally, Gold Read Buffer was loaded onto the plate and the plate was read on the MESO QuickPlex SQ 120MM.

### Statistics

All quantitative measures are compared using one-way ANOVA with Tukey's post-hoc test or Kruskal-Wallis with Dunn's post-hoc test for immunohistochemical (IHC) and western blot (WB) analyses and two-way repeated-measure ANOVA with Bonferroni post-hoc test to analyze behavioral data over a time course. For consistency, any animals with BMS scores of 4 and above 48 hours after SCI were considered unsuccessfully injured and removed from the study. Any subjects not surviving the full length of the study were removed from all analyses. The null hypothesis is rejected if p<0.05. All data was analyzed using GRAPHPAD PRISM® (graphing software) v9.

### Blinding Procedures

The inventors practiced an experimental design that holds several levels of randomization and blinding procedures. First, experimenter #1 assigns mice to the appropriate groups prior to assessing the functional baseline. Experimenters ensure each experimental group has mice of similar ages and weight. All mice will have weights that are within 10% of the average to minimize confounding variables. Second, experimenters #2 and #3 perform surgeries without knowing the cohort the mice are in, while experimenter #4 prepares the syringes for injection, disguising the name of each cohort as letters, for example, labeled A instead of compound name. Finally, experimenter #5 performs the injections. Whenever possible, a mix of animals from all cohorts will be housed together. All behavioral tests were performed by observers blinded to the cohort organization and quantified by different observers also blinded to the groups. Unblinding takes place only after acquiring and analyzing all the data.

### Example 2

### Compounds and Methods for Medical Use

Embodiments of the present disclosure are encompassed in the present example.

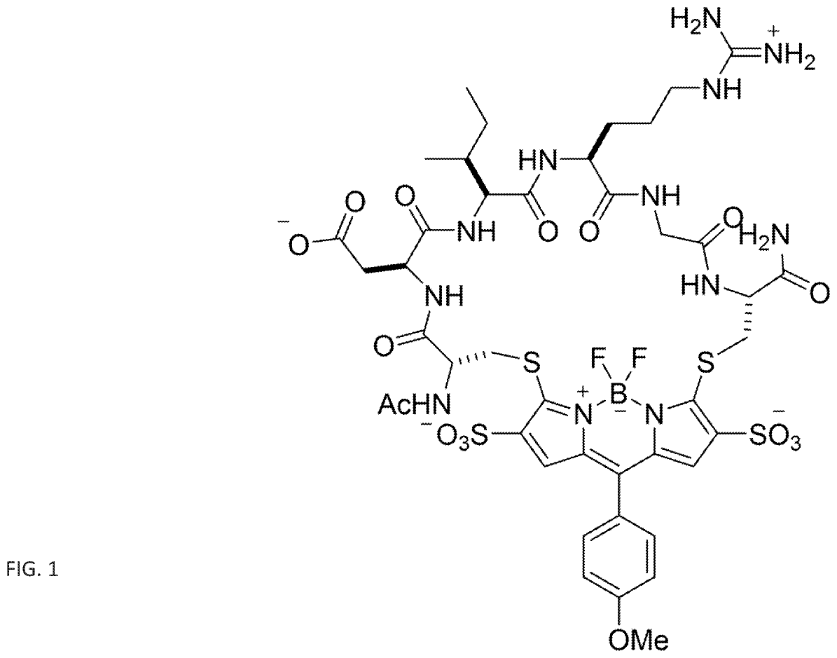

FIG. 1 demonstrates the structure of the exemplary compound 5c(i) (also see PCT Patent Publication WO2022256394). Any of the compounds disclosed herein can be synthesized based on the general procedure provided in PCT Patent Publication WO2022256394.

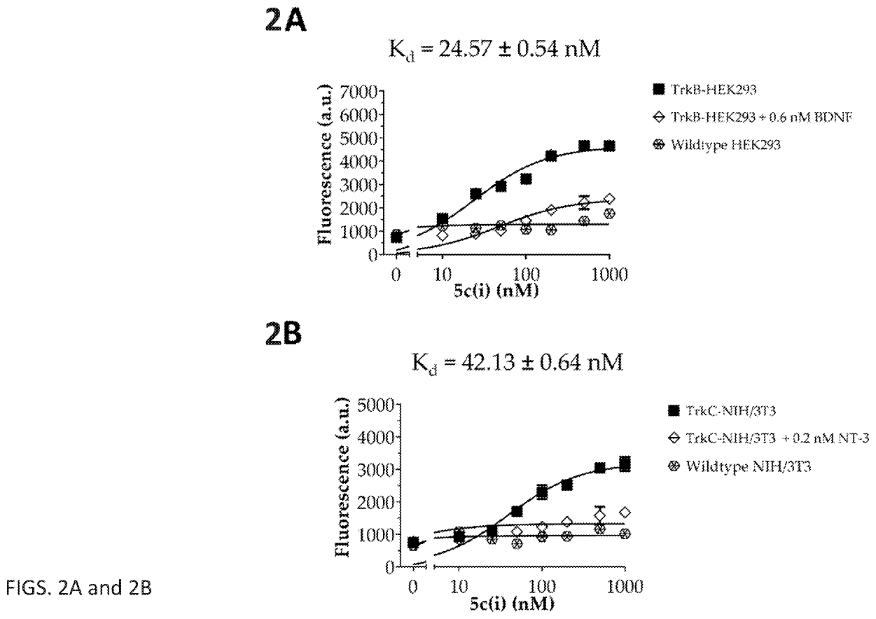

Using TrkB-HEK293 and TrkC-NIH/3T3 cell lines, it was demonstrated that 5c(i) is a highly specific TrkB and TrkC modulator with low nanomolar affinity, mimicking BDNF and NT-3 activity (FIG. 2). Specificity is suggested by lack of binding and biological activity in cells lacking TrkB/TrkC receptors. There is a dire need for TrkB/TrkC modulators because neurotrophins, such as BDNF, are not therapeutically viable because of poor pharmacokinetic profiles⁵⁶. Unlike other pan-Trk modulators^{61,62}, 5c(i) is not designed to bind to the infamous p75 “death receptor” that induces neuronal death⁶³, which is considered in some embodiments to provide increased neuroprotection over other Trk modulators.

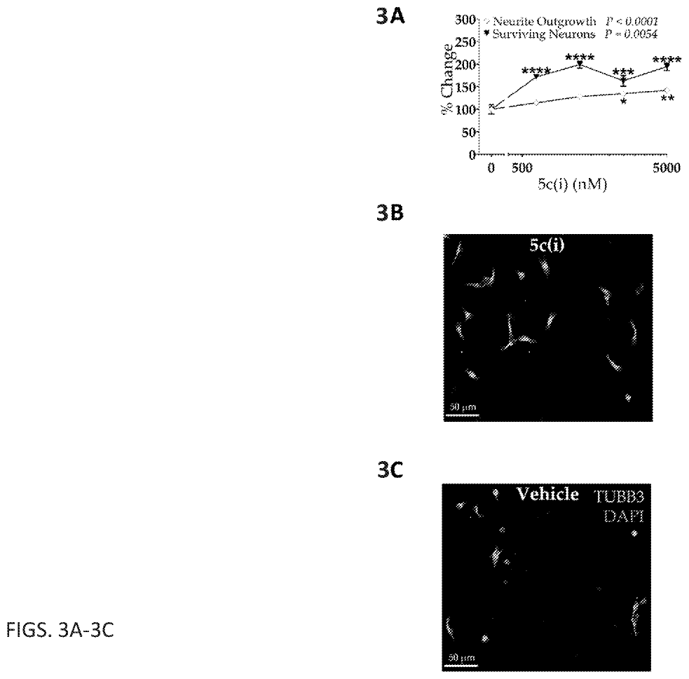

FIG. 3 demonstrates that promoting neuron survival and neurite growth are effective strategies in promoting functional recovery after injury^{64,65}. Neurite regeneration is crucial for memory reconstruction; compounds enhancing neuritogenesis have demonstrated to promote cognitive function in AD models^{66,67}. Culturing adult neurons from 6-month-old mice with 5c(i) for 2 days, as previously described⁵⁸, increased the survival and neurite outgrowth by 94% and 42%, respectively. Therefore, 5c(i) has indications in SCI, AD, and all other diseases neuroprotection and neurite regeneration provide therapeutic value, such as stroke⁶⁸.

Synapsin I, required for hippocampal LTP⁶⁹, is reduced in the dentate gyrus of human AD patients^{70,71}. Astrocytes can release the synaptic protein Synapsin I to provide neuroprotection against oxidation and ischemia and stimulate neurite growth^{72,73}, which can help enhance neuronal survival and LTP in AD^{69,74-77}. Reducing Complement component 3 (C3) levels has effectively promoted functional recovery after SCI^{78,79}. Reducing C3 levels effectively mitigates AD induced synapse loss and neurodegeneration in mice⁸⁰. Reducing astrocytic glycogen synthase kinase-3β (GSK-3β) activity promotes axon regeneration and functional recovery after SCI^{81,82}. GSK-3β impairs LTP, neurogenesis, and memory formation⁸³⁻⁸⁵. Inhibiting GSK-3β expression reduced AP, NFT, neuronal death, and memory deficits in AD models⁸⁹⁻⁸⁸. Altogether, there are many therapeutic advantages in modulating astrocytes to increase Synapsin I expression and reduce C3 and GSK-3β expression.

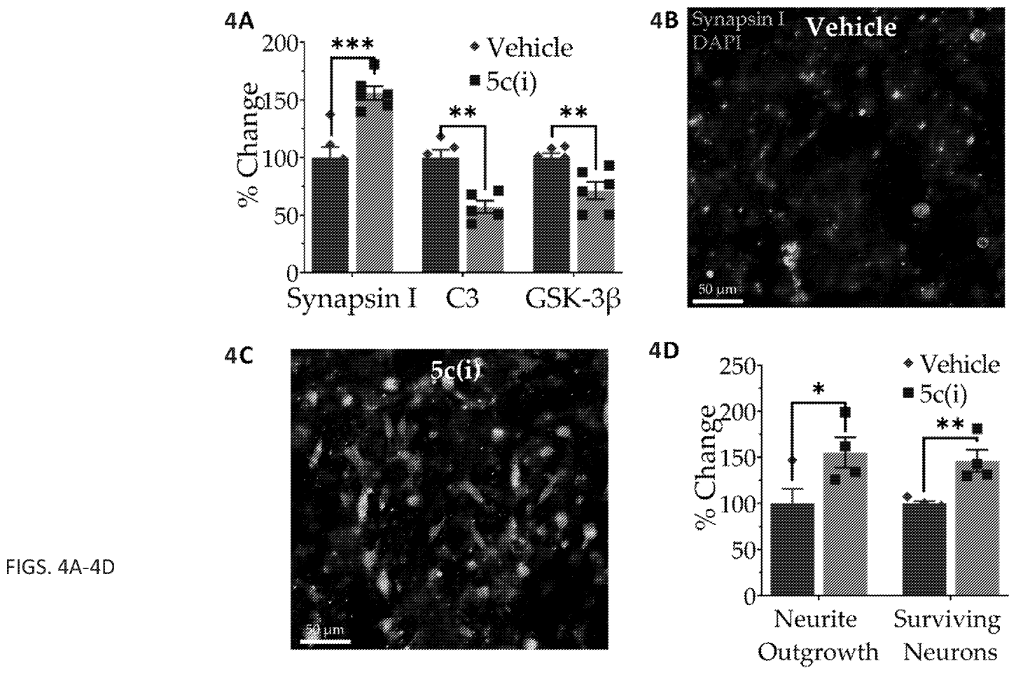

To determine if 5c(i) can modulate astrocytes, adult astrocytes from 6-month-old male mice were treated with 5c(i) for 3 days and stained with Synapsin I and C3 to measure changes in fluorescent intensity or western blot was conducted on the cell lysate to assess changes in GSK-3β expression (FIG. 4). This resulted in a significant increase in Synapsin I production and reduction of C3 and GSK-3β expression. To determine if 5c(i) treated astrocytes can directly benefit neuronal recovery, astrocytes were cultured with 5c(i) for 2 days before removing 5c(i) from the culture and plating adult neurons on top of the astrocytes for 2DIV, resulting in increased neuron survival and neurite growth. Therefore, 5c(i) can modulate astrocytes to increase Synapsin I release to support synaptic plasticity, reduce the expression of neuroinflammatory agents C3 and GSK-3β to reduce neurotoxicity, and prime the astrocytes to increase neuron survival and neurite growth, altogether, hypothesized to enhance neurological recovery. Therefore, 5c(i) has indications in SCI, AD, and all other diseases for modulation of astrocytes, C3 expression, and GSK-3β expression to provide therapeutic value.

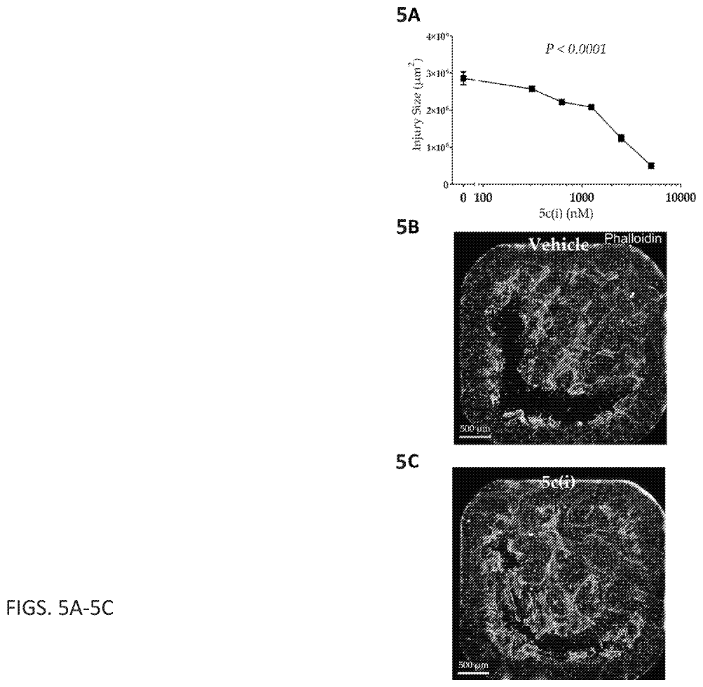

FIG. 5 demonstrates that promotion of astrocyte migration can reduce the lesion size and promote functional recovery after SCI⁸¹. Astrocyte polarization, which is the extension of processes towards an insult and usually beneficial, is decreased in AD, leading to reduced clearance of Aβ plaques^{90,91}. Therefore, a scratch assay using a glial astrocyte culture was conducted to determine if this effect could be replicated using 5c(i) in adult astrocytes. Astrocytes from 6-month-old female mice were cultured until full confluency was reached. The plate was then scratched to mimic the neural cell death and neuro-toxic debris accumulation found after SCI^{92,93}. Immediately after scratching, 5c(i) was added for 48 hours which resulted in the artificial wound closing at an expedited rate. Therefore, 5c(i) has indications at least in SCI, AD, and all other diseases where astrocyte migration and polarization provide therapeutic value.

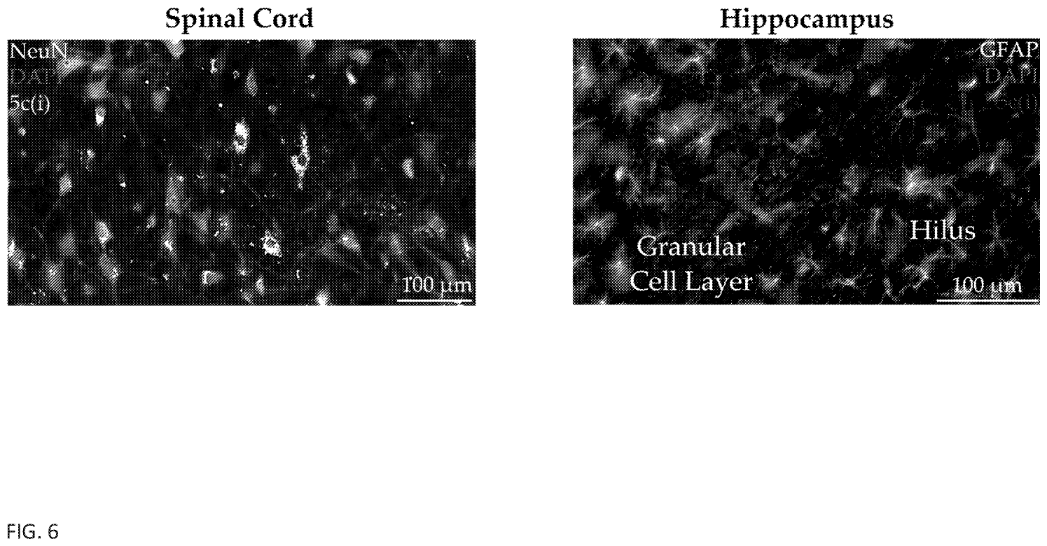

The compound 5c(i), an intrinsically fluorescent compound, was found to easily penetrate the blood-brain barrier (BBB) and blood-spinal cord barrier (BSCB) through intraperitoneal (IP) administration to target neurons and other neural cells, even in uninjured animals without increased BBB or BSCB permeability (FIG. 6). This is highly significant as most drugs cannot penetrate the central nervous system (CNS), resulting in very costly late-stage failures in CNS drug development⁹⁴. 5c(i) therefore in specific embodiments can cross the BBB and target the brain, such as through IP administration, for indications relating to brain function, such as AD and stroke. 5c(i) has even higher potential for penetrating the CNS after SCI when permeability is increased⁹⁵⁻⁹⁷.

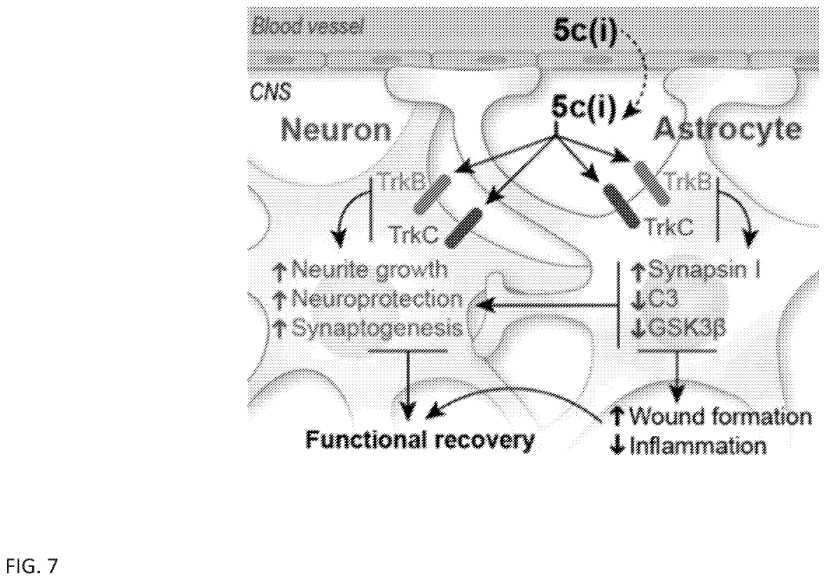

FIG. 7 provides a graphical representation of the activity of 5c(i) (as one example of a compound) on neurons and astrocytes.

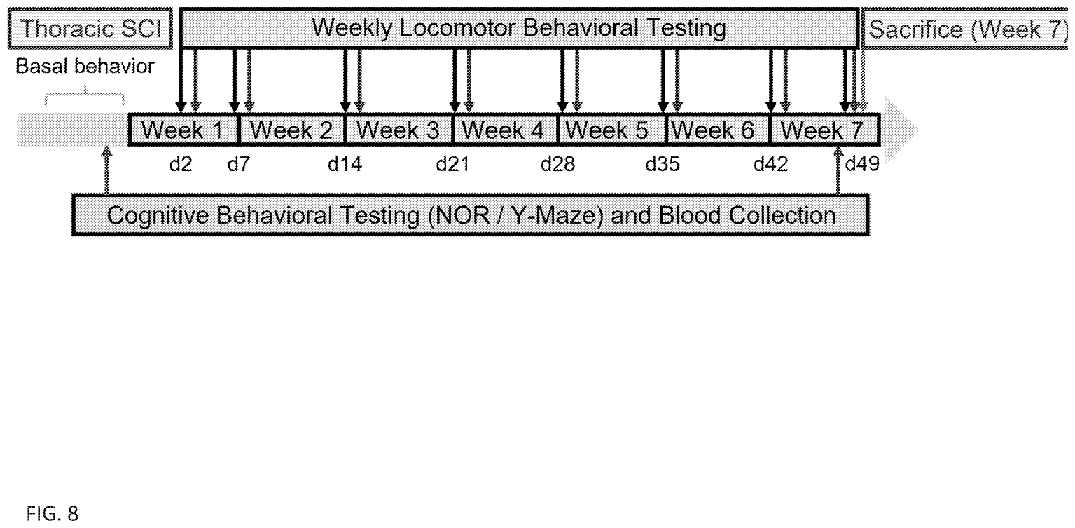

In FIG. 8, mice underwent baseline cognitive testing and blood collection immediately before SCI, and one embodiment of such a process is provided therein. Afterwards, mice sustained an SCI at 8 weeks of age. SCI model: thoracic-8 contusion (50 kdyne impact) and compression (2-see dwell time), a severe contusion/compression model that accurately represents injuries seen in the clinic. 5c(i) (5 or 25 mg/kg/day) or Vehicle were administered IP daily starting 4 hours post injury to reflect the delay found in the clinic. Locomotor behavioral testing (BMS) was conducted weekly for 7 weeks. 7 weeks after injury, the mice underwent cognitive behavioral testing (NOR) and blood collection once again. Mice were euthanized 7 weeks after sustaining an SCI and underwent perfusion and tissue preparations for further analysis.

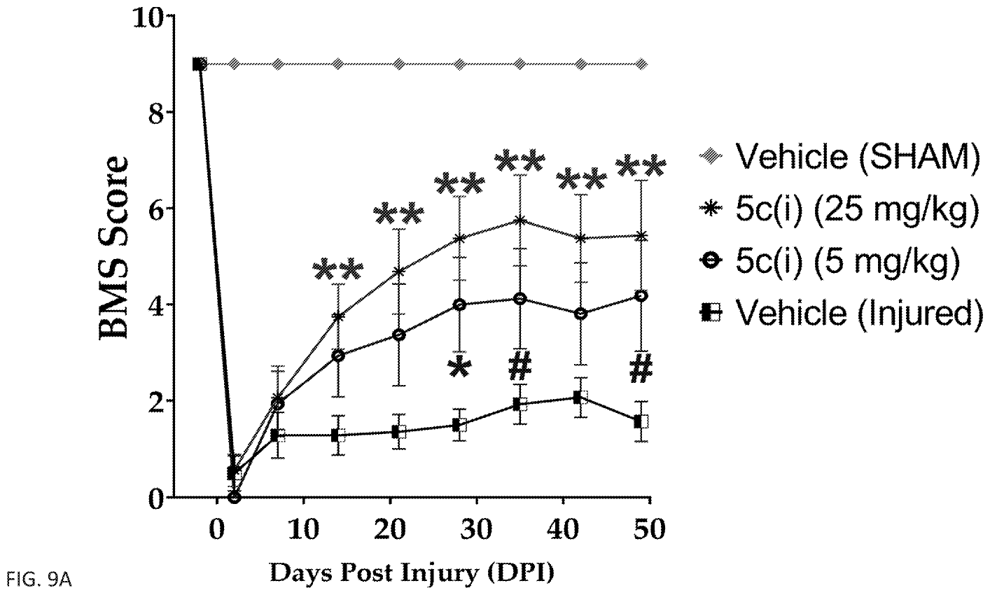

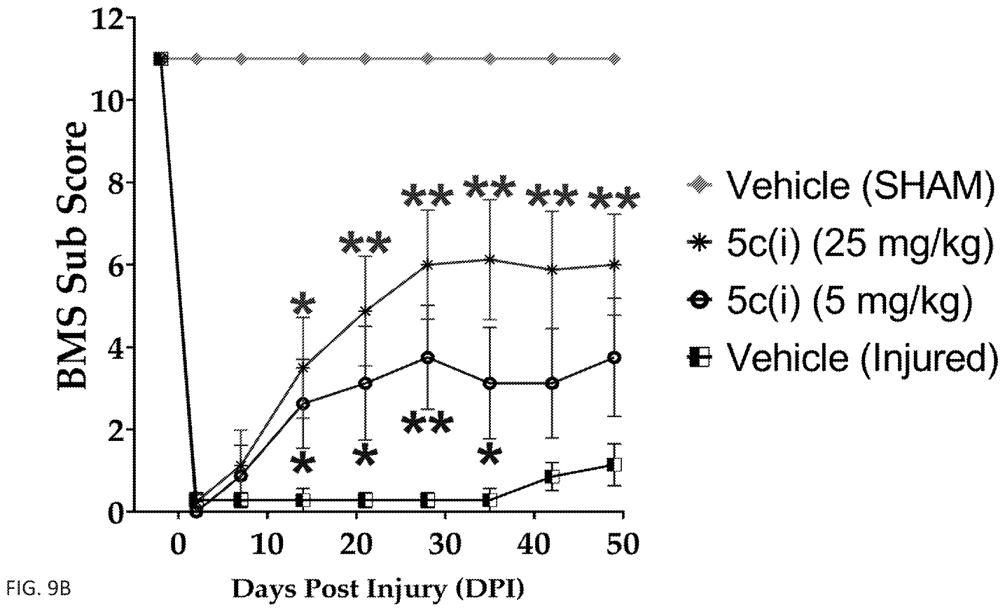

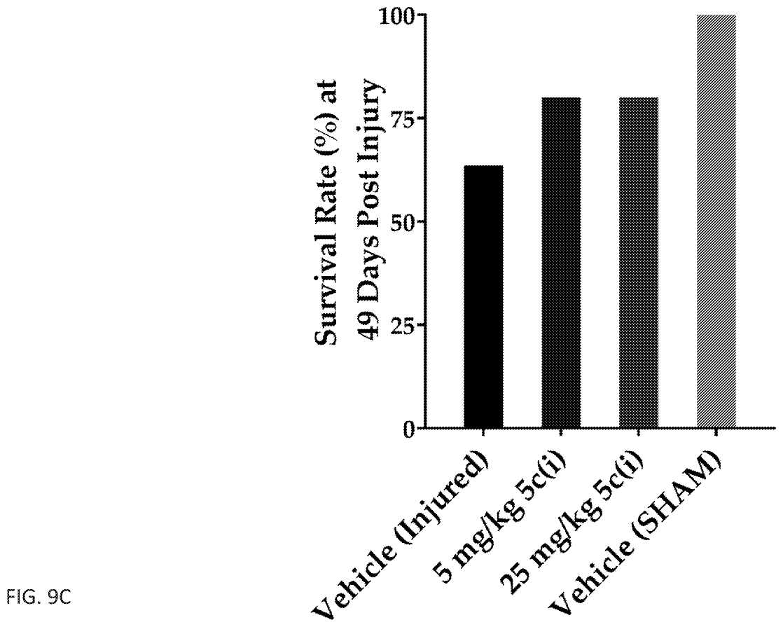

For locomotor behavioral testing, BMS tests were performed (FIG. 9). SCI induces paralysis that results in lower BMS scores. For BMS and BMS Sub scores, no significant differences between injured cohorts were found at 2 and 7 days post injury (DPI), signifying that all treatment groups received injuries of a similar degree. 5c(i) significantly improved the BMS and BMS Sub scores after treatment. Therefore, 5c(i) has indications in cases that result in paralysis or reduced locomotor function.

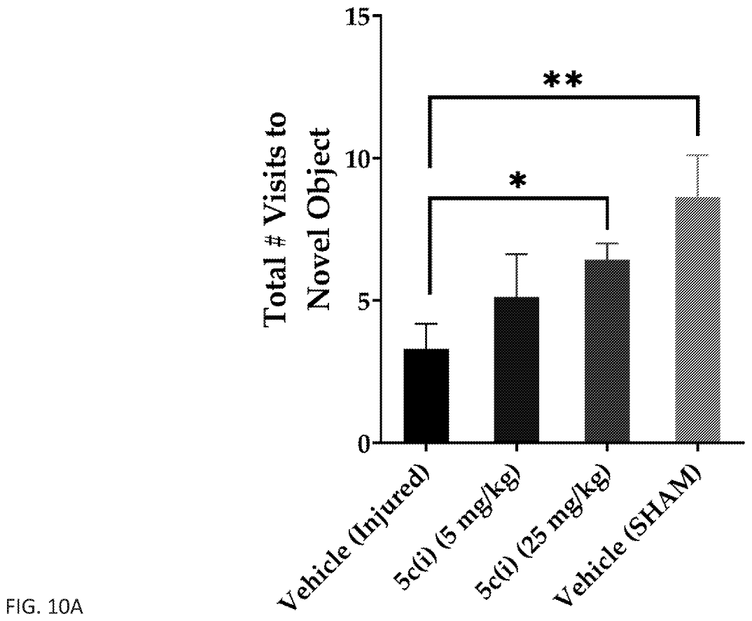

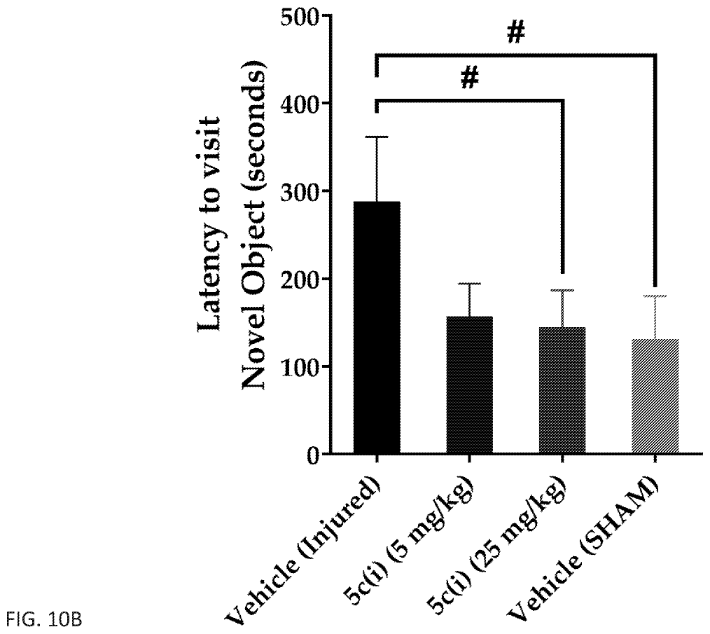

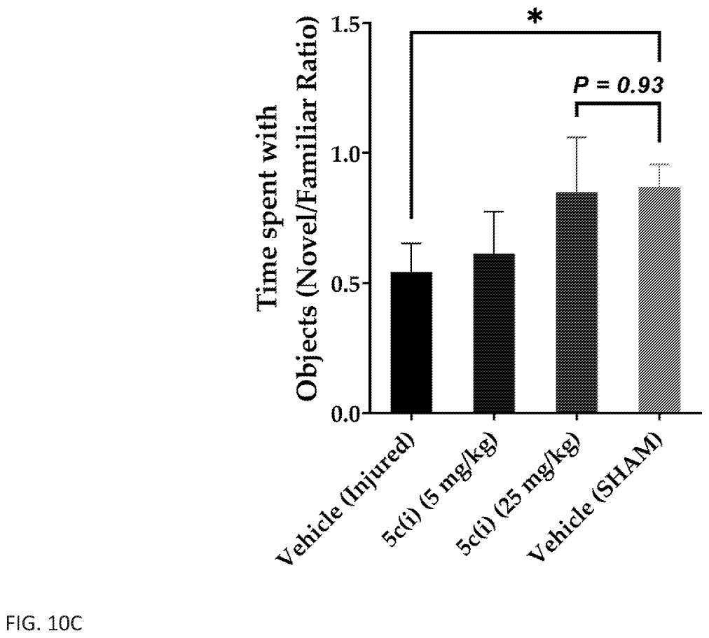

Patients with neurodegenerative diseases and neurotrauma suffer from cognitive impairments. Since SCI can reduce cognitive function⁵, hippocampal-dependent learning and memory was assessed in the SCI mice using the Novel Object Recognition (NOR) test (using a 24-hr probe/test interval)⁹⁸⁻¹⁰⁰ at 7 weeks post injury. Treatment with 5c(i) resulted in more visits, reduced latency to visit, and increased preference for the novel object, performing similar to the uninjured mice (FIG. 10). This data indicates that in particular embodiments 5c(i) can improve long-term memory. Therefore, 5c(i) is useful to improve memory function in patients, such as with cognitive dysfunction and thus, 5c(i) has indications in all diseases that reduce memory function.

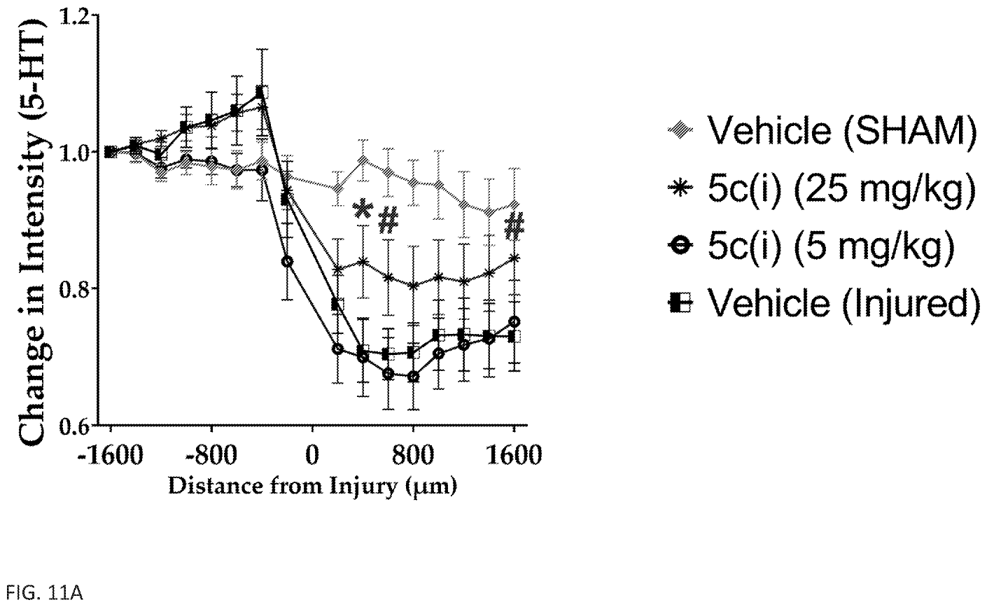

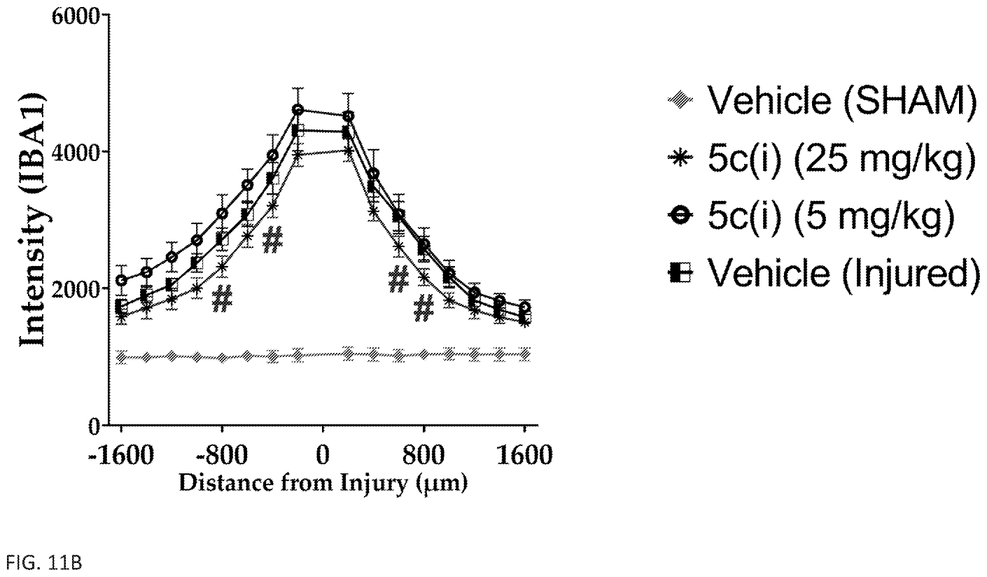

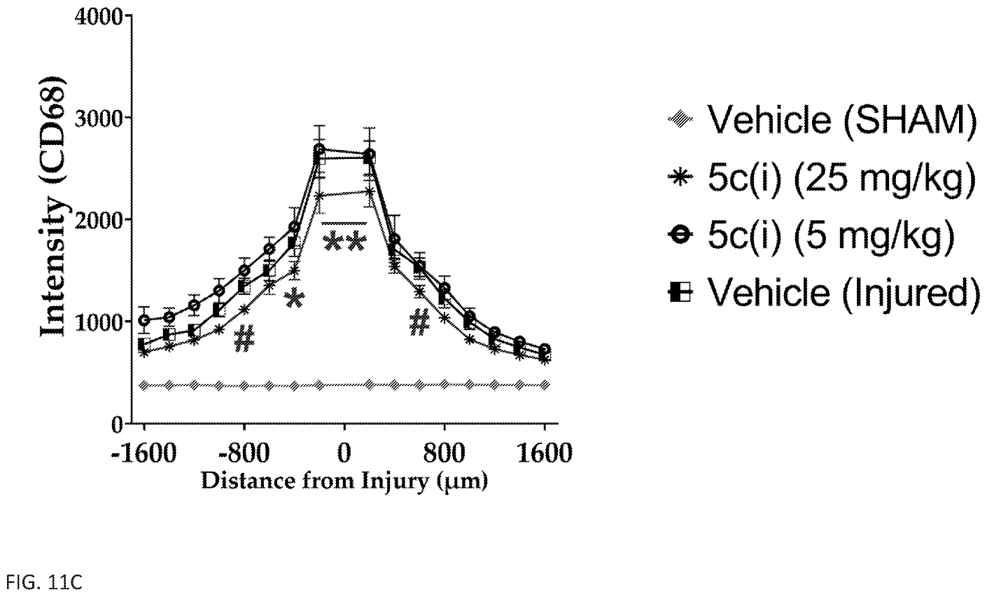

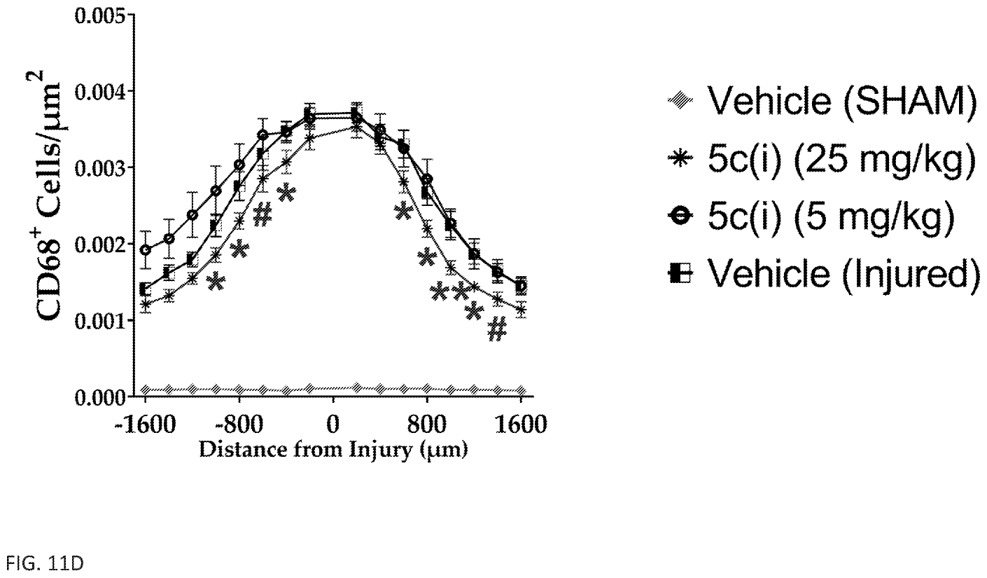

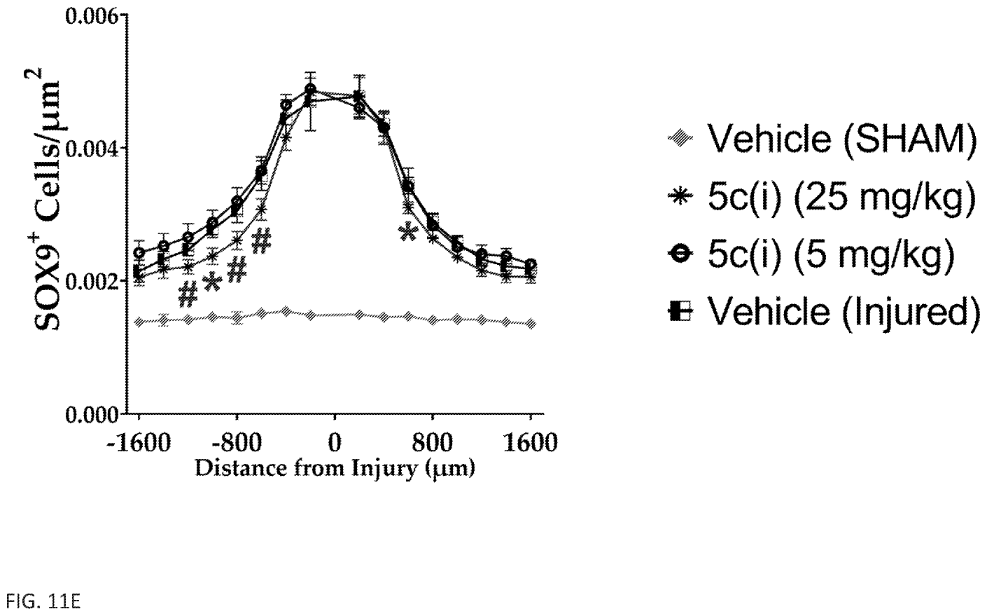

Histological analysis of the spinal cord was conducted 7 weeks post injury (FIG. 11). 5c(i) increased serotonergic (5-HT) axon immunoreactivity caudal to the injury site, indicating 5c(i) enhanced axon regeneration, survival, and/or sprouting¹⁰¹. 5c(i) reduced IBA1 immunoreactivity implying a reduction in microglial activation.^{102,103} Chronic microglial activation is associated with neurodegeneration and cognitive dysfunction^{104,105}, therefore, a reduction in the chronic phases, as seen here, is highly beneficial. 5c(i) significantly reduced CD68 immunoreactivity at the injury site and prevalence of CD68⁺ (mononuclear phagocyte¹⁰⁶) cells around the injury site, implying 5c(i) has anti-inflammatory and anti-cancer properties^{106,107} and may prevent cognitive impairments^{108,109}. 5c(i) reduced the number of SOX9′ cells around the injury site which is expected to mitigate the risk of cancer¹¹⁰⁻¹¹², reduce chondroitin sulfate proteoglycan (CSPG) deposition and collagenous scarring at the lesion. Therefore, 5c(i) has indications in diseases relating to regeneration of tissue, cells, and other organic matter, inflammatory diseases, metabolic disease, cancer, cognitive impairments, and diseases relating to CSPG and collagenous scarring.

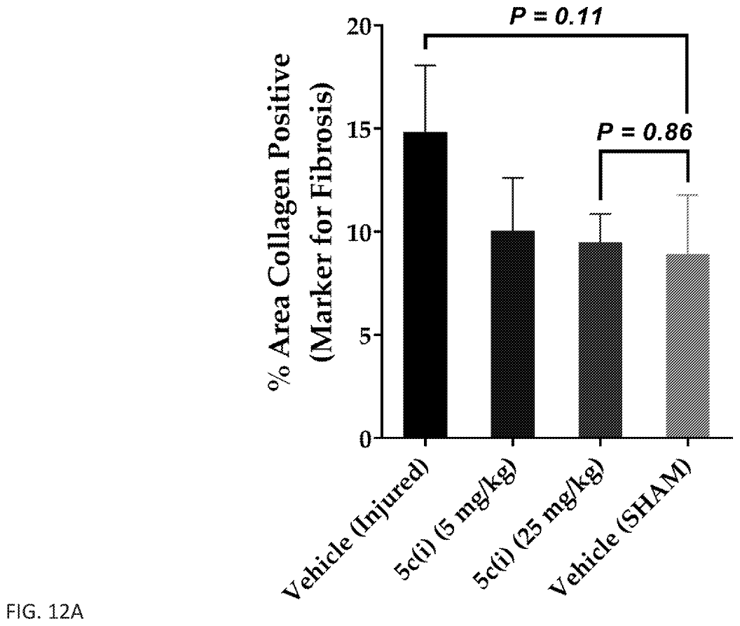

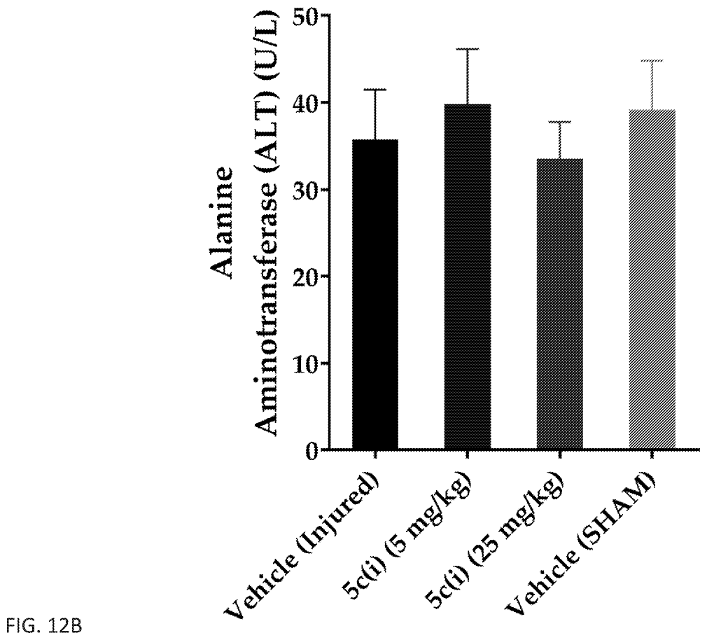

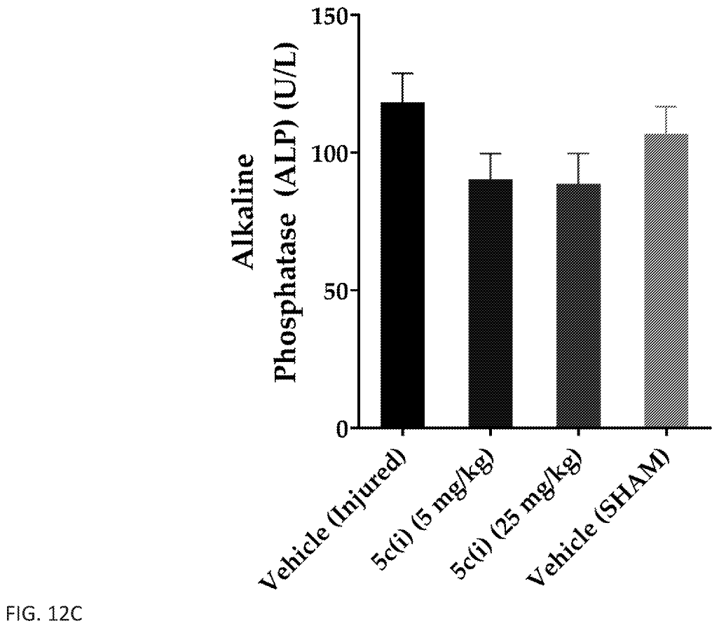

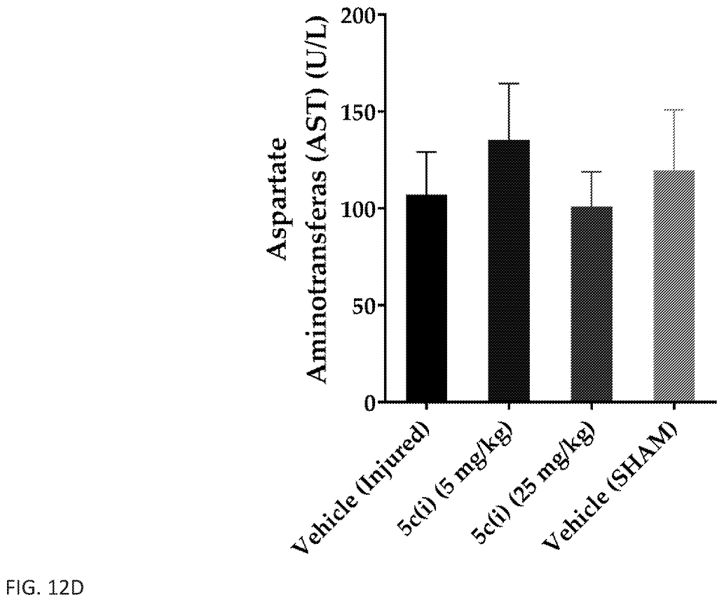

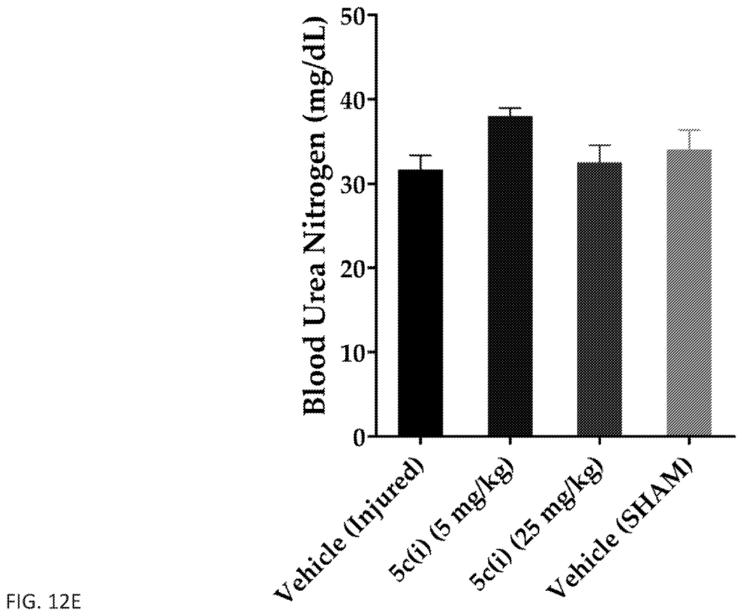

Seven weeks after injury, histological analysis was performed on livers, and toxicology screens were conducted on plasma (FIG. 12). 5c(i) alleviated collagen deposition, a marker for liver fibrosis the inventors found present in mice with SCI¹, therefore, in specific embodiments 5c(i) mitigates liver pathologies, such as nonalcoholic fatty liver disease, found in human SCI patients^{113,114}. Therefore, 5c(i) has indications in diseases affecting liver function, in particular embodiments. Assessment of plasma at 7 weeks post injury revealed 5c(i) does not alter liver function enzymes alanine aminotransferase (ALT), alkaline phosphatase (ALP), and aspartate aminotransferase (AST)¹¹⁵ nor docs it alter the kidney function marker blood urea nitrogen (BUN)¹¹⁶.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1 National Spinal Cord Injury Statistical Center. Facts and Figures at a Glance, <https://www.nscisc.uab.edu/> (2021).
2 Christopher & Dana Reeve Foundation. Prevalence of paralysis in the United States, <https://www.christopherreeve.orgfliving-with-paralysis/stats-about-paralysis> (2021).
3 Craig, A., Guest, R., Tran, Y. & Middleton, J. Cognitive Impairment and Mood States after Spinal Cord Injury. J Neurotrauma 34, 1156-1163, doi:10.1089/neu.2016.4632 (2017).
4 Macciocchi, S. N., Seel, R. T. & Thompson, N. The impact of mild traumatic brain injury on cognitive functioning following co-occurring spinal cord injury. Arch Clin Neuropsychol 28, 684-691, doi:10.1093/arclin/act049 (2013).
5 Sefiani, A. & Geoffroy, C. G. The Potential Role of Inflammation in Modulating Endogenous Hippocampal Neurogenesis After Spinal Cord Injury. Front Neurosci 15, 682259, doi:10.3389/fnins.2021.682259 (2021).
6 Alcántar-Garibay, O. V., Incontri-Abraham, D. & Ibarra, A. Spinal cord injury-induced cognitive impairment: a narrative review. Neural Regen Res 17, 2649-2654, doi:10.4103/1673-5374.339475 (2022).
7 Wilmot, C. B., Cope, D. N., Hall, K. M. & Acker, M. Occult head injury: its incidence in spinal cord injury. Arch Phys Med Rehabil 66, 227-231, doi:10.1016/0003-9993(85)90148-0 (1985).
8 Chiaravalloti, N. D., Weber, E., Wylie, G., Dyson-Hudson, T. & Wecht, J. M. Patterns of cognitive deficits in persons with spinal cord injury as compared with both age-matched and older individuals without spinal cord injury. The journal of spinal cord medicine 43, 88-97. doi:10.1080/10790268.2018.1543103 (2020).
9 Cheung, V., Hoshide, R., Bansal, V., Kasper, E. & Chen, C. C. Methylprednisolone in the management of spinal cord injuries: Lessons from randomized, controlled trials. Surg Neurol Int 6, 142, doi:10.4103/2152-7806.163452 (2015).
10 Yoon, D. H., Kim, Y. S. & Young, W. Therapeutic time window for methylprednisolone in spinal cord injured rat. Yonsei Med J 40, 313-320, doi:10.3349/ymj.1999.40.4.313 (1999).
11 Lee, D. Y. et al. The Importance of Early Surgical Decompression for Acute Traumatic Spinal Cord Injury. Clin Orthop Surg 10, 448-454, doi:10.4055/cios.2018.10.4.448 (2018).
12 Ghnenis, A. B. et al. Evaluation of the Cardiometabolic Disorders after Spinal Cord Injury in Mice. Biology 11, 495 (2022).
13 Sefiani, A. & Geoffroy, C. G. The Potential Role of Inflammation in Modulating Endogenous Hippocampal Neurogenesis After Spinal Cord Injury. Frontiers in Neuroscience 15, doi:10.3389/fnins.2021.682259 (2021).
14 Sharma, N. & Singh, A. N. Exploring Biomarkers for Alzheimer's Disease. J Clin Diagn Res 10, Ke01-06, doi:10.7860/jcdr/2016/18828.8166 (2016).
15 Kashyap, G. et al. Synapse loss and progress of Alzheimer's disease—A network model. Scientific Reports 9, 6555, doi:10.1038/s41598-019-43076-y (2019).
16 Rajan, K. B. et al. Population estimate of people with clinical Alzheimer's disease and mild cognitive impairment in the United States (2020-2060). Alzheimers Dement 17, 1966-1975, doi:10.1002/alz.12362 (2021).
17 Wong, W. Economic burden of Alzheimer disease and managed care considerations. Am J Manag Care 26, S177-s183, doi:10.37765/ajmc.2020.88482 (2020).
18 iHealthcareAnalyst. Global Alzheimer's Disease Market $25 Billion by 2029. (2022).
19 Olivares, D. et al. N-methyl D-aspartate (NMDA) receptor antagonists and memantine treatment for Alzheimer's disease, vascular dementia and Parkinson's disease. Curr Alzheimer Res 9, 746-758, doi:10.2174/156720512801322564 (2012).
20 Russ, T. C. & Morling, J. R. Cholinesterase inhibitors for mild cognitive impairment. Cochrane Database Syst Rev 2012, Cd009132, doi:10.1002/14651858.CD009132.pub2 (2012).
21 Yang, Z., Zhou, X. & Zhang, Q. Effectiveness and safety of memantine treatment for Alzheimer's disease. J Alzheimers Dis 36, 445-458, doi:10.3233/jad-130395 (2013).
22 Bradford, A., Kunik, M. E., Schulz, P., Williams, S. P. & Singh, H. Missed and delayed diagnosis of dementia in primary care: prevalence and contributing factors. Alzheimer Dis Assoc Disord 23, 306-314, doi:10.1097/WAD.0b013e3181a6bebc (2009).
23 Rasmussen, J. & Langerman, H. Alzheimer's Disease—Why We Need Early Diagnosis. Degener Neurol Neuromuscul Dis 9, 123-130, doi:10.2147/dnnd.S228939 (2019).
24 Alzheimer's Association. 2015 Alzheimer's Disease Facts and Figures, <https://www.alz.org/media/documents/2015factsandfigures.pdf> (2015).
25 Ma, L. et al. Yizhi Qingxin Formula Extract Ameliorates Cognitive Decline in Aged Rats via the Brain-Derived Neurotrophic Factor/Tropomyosin Receptor Kinase B Pathway. Frontiers in Pharmacology 11, doi:10.3389/fphar.2020.00510 (2020).
26 Mantilla, C. B., Greising, S. M., Stowe, J. M., Zhan, W. Z. & Sieck, G. C. TrkB kinase activity is critical for recovery of respiratory function after cervical spinal cord hemisection. Exp Neurol 261, 190-195, doi:10.1016/j.expneurol.2014.05.027 (2014).
27 Han, J. et al. Delayed administration of a small molecule tropomyosin-related kinase B ligand promotes recovery after hypoxic-ischemic stroke. Stroke 43, 1918-1924, doi:10.1161/strokeaha.111.641878 (2012).
28 Tejeda, G. S. & Díaz-Guerra, M. Integral Characterization of Defective BDNF/TrkB Signalling in Neurological and Psychiatric Disorders Leads the Way to New Therapies. Int J Mol Sci 18, doi:10.3390/ijms18020268 (2017).
29 Mantilla, C. B., Gransee, H. M., Zhan, W. Z. & Sieck, G. C. Motoneuron BDNF/TrkB signaling enhances functional recovery after cervical spinal cord injury. Exp Neurol 247, 101-109, doi:10.1016/j.expneurol.2013.04.002 (2013).
30 Chen, C. et al. Optimized TrkB Agonist Ameliorates Alzheimer's Disease Pathologies and Improves Cognitive Functions via Inhibiting Delta-Secretase. ACS Chem Neurosci 12, 2448-2461, doi:10.1021/acschemneuro.1c00181 (2021).
31 Proenca, C. C., Song, M. & Lee, F. S. Differential effects of BDNF and neurotrophin 4 (NT4) on endocytic sorting of TrkB receptors. J Neurochem 138, 397-406, doi:10.1111/jnc.13676 (2016).
32 Erickson, K. I. et al. Brain-derived neurotrophic factor is associated with age-related decline in hippocampal volume. The Journal of neuroscience: the official journal of the Society for Neuroscience 30, 5368-5375, doi:10.1523/JNEUROSCI.6251-09.2010 (2010).
33 Bathina, S. & Das, U. N. Brain-derived neurotrophic factor and its clinical implications. Archives of medical science: AMS 11, 1164-1178, doi:10.5114/aoms.2015.56342 (2015).
34 Casarotto, P. C. et al. Antidepressant drugs act by directly binding to TRKB neurotrophin receptors. Cell 184, 1299-1313.e1219. doi:10.1016/j.cell.2021.01.034 (2021).
35 Crowley, S. T., Fukushima, Y., Uchida, S., Kataoka, K. & Itaka, K. Enhancement of Motor Function Recovery after Spinal Cord Injury in Mice by Delivery of Brain-Derived Neurotrophic Factor mRNA. Mol Ther Nucleic Acids 17, 465-476, doi:10.1016/j.omtn.2019.06.016 (2019).
36 Liang, J., Deng, G. & Huang, H. The activation of BDNF reduced inflammation in a spinal cord injury model by TrkB/p38 MAPK signaling. Experimental and therapeutic medicine 17, 1688-1696, doi:10.3892/etm.2018.7109 (2019).
37 Stucky, C. L., Shin, J. B. & Lewin, G. R. Neurotrophin-4: a survival factor for adult sensory neurons. Curr Biol 12, 1401-1404, doi:10.1016/s0960-9822(02)01072-2 (2002).
38 English, A. W., Cucoranu, D., Mulligan, A., Rodriguez, J. A. & Sabatier, M. J. Neurotrophin-4/5 is implicated in the enhancement of axon regeneration produced by treadmill training following peripheral nerve injury. The European journal of neuroscience 33, 2265-2271, doi:10.1111/j.1460-9568.2011.07724.x (2011).
39 Liu, J. et al. Effects of NT-4 gene modified fibroblasts transplanted into AD rats. Neurosci Lett 466, 1-5, doi:10.1016/j.neulet.2009.09.020 (2009).
40 Wang, T. et al. NT-4 attenuates neuroinflammation via TrkB/PI3K/FoxO1 pathway after germinal matrix hemorrhage in neonatal rats. Journal of Neuroinflammation 17, 158, doi:10.1186/s12974-020-01835-z (2020).
41 Endres, M. et al. Ischemic brain damage in mice after selectively modifying BDNF or NT4 gene expression. J Cereb Blood Flow Metab 20, 139-144, doi:10.1097/00004647-200001000-00018 (2000).
42 Endres, M., Fan, G., Hirt, L. & Jaenisch, R. Stroke damage in mice after knocking the neutrophin-4 gene into the brain-derived neurotrophic factor locus. J Cereb Blood Flow Metab 23, 150-153, doi:10.1097/01.Web.0000043949.67811.C6 (2003).
43 Chen, C. et al. Optimized TrkB Agonist Ameliorates Alzheimer's Disease Pathologies and Improves Cognitive Functions via Inhibiting Delta-Secretase. ACS Chemical Neuroscience 12, 2448-2461, doi:10.1021/acschemneuro.1c00181 (2021).
44 Wang, S. et al. Therapeutic potential of a TrkB agonistic antibody for Alzheimer's disease. Theranostics 10, 6854-6874, doi:10.7150/thno.44165 (2020).
45 Fouad, K., Vavrek, R. & Cho, S. A TrkB Antibody Agonist Promotes Plasticity after Cervical Spinal Cord Injury in Adult Rats. J Neurotrauma 38, 1338-1348, doi:10.1089/neu.2009.1116 (2021).
46 Li, X. et al. Exercise training modulates glutamic acid decarboxylase-65/67 expression through TrkB signaling to ameliorate neuropathic pain in rats with spinal cord injury. Molecular pain 16, 1744806920924511-1744806920924511, doi:10.1177/1744806920924511 (2020).
47 Lamballe, F., Tapley, P. & Barbacid, M. trkC encodes multiple neurotrophin-3 receptors with distinct biological properties and substrate specificities. The EMBO journal 12, 3083-3094, doi:10.1002/j.1460-2075.1993.tb05977.x (1993).
48 Kaisho, Y., Shintani, A., Nishida, M., Fukumoto, H. & Igarashi, K. Developmental changes of neurotrophin-3 level in the mouse brain detected by a highly sensitive enzyme immunoassay. Brain Research 666, 143-146, doi:https://doi.org/10.1016/0006-8993(94)90297-6 (1994).
49 Hory-Lee, F., Russell, M., Lindsay, R. M. & Frank, E. Neurotrophin 3 supports the survival of developing muscle sensory neurons in culture. Proceedings of the National Academy of Sciences of the United States of America 90, 2613-2617, doi:10.1073/pnas.90.7.2613 (1993).
50 Francis, N. et al. NT-3, like NGF, is required for survival of sympathetic neurons, but not their precursors. Dev Biol 210, 411-427, doi:10.1006/dbio.1999.9269 (1999).
51 McTigue, D. M., Homer. P. J., Stokes, B. T. & Gage, F. H. Neurotrophin-3 and brain-derived neurotrophic factor induce oligodendrocyte proliferation and myelination of regenerating axons in the contused adult rat spinal cord. J Neurosci 18, 5354-5365, doi:10.1523/jneurosci.18-14-05354.1998 (1998).
52 Bao, S.-S. et al. NT3 treatment alters spinal cord injury-induced changes in the gray matter volume of rhesus monkey cortex. Scientific Reports 12, 5919, doi:10.1038/s41598-022-09981-5 (2022).
53 Yan, Z. et al. Neurotrophin-3 Promotes the Neuronal Differentiation of BMSCs and Improves Cognitive Function in a Rat Model of Alzheimer's Disease. Front Cell Neurosci 15, 629356, doi:10.3389/fncel.2021.629356 (2021).
54 Ye, J. et al. Effect of NT-3 on repair of spinal cord injury through the MAPK signaling pathway. Eur Rev Med Pharmacol Sci 24, 2165-2172. doi:10.26355/eurrev_202003_20481 (2020).
55 Li, G. et al. An NT-3-releasing bioscaffold supports the formation of TrkC-modified neural stem cell-derived neural network tissue with efficacy in repairing spinal cord injury. Bioactive materials 6, 3766-3781, doi:10.1016/j.bioactmat.2021.03.036 (2021).
56 Jang, S.-W. et al. A selective TrkB agonist with potent neurotrophic activities by 7,8-dihydroxyflavone. Proceedings of the National Academy of Sciences 107, 2687-2692, doi:doi:10.1073/pnas.0913572107 (2010).
57 Poduslo, J. F. & Curran, G. L. Permeability at the blood-brain and blood-nerve barriers of the neurotrophic factors: NGF, CNTF, NT-3, BDNF. Molecular Brain Research 36, 280-286, doi:https://doi.org/10.1016/0169-328X(95)00250-V (1996).
58 Sefiani, A., Rusyn, I. & Geoffroy, C. G. Novel Adult Cortical Neuron Processing and Screening method illustrates Sex- and Age-dependent effects of pharmaceutical compounds. bioRxiv, 2022.2003.2029.486299, doi:10.1101/2022.03.29.486299 (2022).
59 Basso, D. M. et al. Basso Mouse Scale for locomotion detects differences in recovery after spinal cord injury in five common mouse strains. J Neurotrauma 23, 635-659, doi:10.1089/neu.2006.23.635 (2006).
60 Hook, M. A. et al. Osteopenia in a Mouse Model of Spinal Cord Injury: Effects of Age, Sex and Motor Function. Biology (Basel) 11, doi:10.3390/biology11020189 (2022).
61 Conroy, J. N. & Coulson, E. J. High-affinity TrkA and p75 neurotrophin receptor complexes: A twisted affair. J Biol Chem 298, 101568, doi:10.1016/j.jbc.2022.101568 (2022).
62 Barcelona, P. F. & Saragovi, H. U. A Pro-Nerve Growth Factor (proNGF) and NGF Binding Protein, β2-Macroglobulin, Differentially Regulates p75 and TrkA Receptors and Is Relevant to Neurodegeneration Ex Vivo and In Vivo. Mol Cell Biol 35, 3396-3408, doi:10.1128/mcb.00544-15 (2015).
63 Barrett, G. L. The p75 neurotrophin receptor and neuronal apoptosis. Prog Neurobiol 61, 205-229, doi:10.1016/s0301-0082(99)00056-8 (2000).
64 Romaus-Sanjurjo, D. et al. GABA promotes survival and axonal regeneration in identifiable descending neurons after spinal cord injury in larval lampreys. Cell Death & Disease 9, 663, doi:10.1038/s41419-018-0704-9 (2018).
65 Shabanzadeh, A. P. et al. Modifying PTEN recruitment promotes neuron survival, regeneration, and functional recovery after CNS injury. Cell Death & Disease 10, 567, doi:10.1038/s41419-019-1802-z (2019).
66 Yang, Z., Song, C., Ge, Y. W. & Tohda, C. Editorial: Treatment of Alzheimer's disease-discovery of natural products based on neurite outgrowth and neuroprotection. Front Pharmacol 13, 1079783, doi:10.3389/fphar.2022.1079783 (2022).
67 Jesky, R. & Chen, H. The neuritogenic and neuroprotective potential of senegenin against Aβ-induced neurotoxicity in PC 12 cells. BMC Complement Altern Med 16, 26, doi:10.1186/s12906-016-1006-3 (2016).
68 Minnerup, J., Sutherland, B. A., Buchan, A. M. & Kleinschnitz, C. Neuroprotection for stroke: current status and future perspectives. Int J Mol Sci 13, 11753-11772, doi:10.3390/ijms130911753 (2012).
69 Kushner, S. A. et al. Modulation of presynaptic plasticity and learning by the H-ras/extracellular signal-regulated kinase/synapsin I signaling pathway. J Neurosci 25, 9721-9734, doi:10.1523/jneurosci.2836-05.2005 (2005).
70 Qin, S., Hu, X. Y., Xu, H. & Zhou, J. N. Regional alteration of synapsin T in the hippocampal formation of Alzheimer's disease patients. Acta Neuropathol 107, 209-215, doi:10.1007/s00401-003-0800-4 (2004).
71 Zhang, L. et al. Rewiring of regenerated axons by combining treadmill training with semaphorin 3A inhibition. Mol Brain 7, 14, doi:10.1186/1756-6606-7-14 (2014).
72 Wang, S. et al. Synapsin I is an oligomannose-carrying glycoprotein, acts as an oligomannose-binding lectin, and promotes neurite outgrowth and neuronal survival when released via glia-derived exosomes. J Neurosci 31, 7275-7290, doi:10.1523/jneurosci.6476-10.2011 (2011).
73 Upadhya, R., Zingg, W., Shetty, S. & Shetty, A. K. Astrocyte-derived extracellular vesicles: Neuroreparative properties and role in the pathogenesis of neurodegenerative disorders. J Control Release 323, 225-239, doi:10.1016/j.jconrel.2020.04.017 (2020).
74 Luca, M., Luca, A. & Calandra, C. The Role of Oxidative Damage in the Pathogenesis and Progression of Alzheimer's Disease and Vascular Dementia. Oxid Med Cell Longev 2015, 504678, doi:10.1155/2015/504678 (2015).
75 Zhu, X. et al. Vascular oxidative stress in Alzheimer disease. J Neurol Sci 257, 240-246, doi:10.1016/j.jns.2007.01.039 (2007).
76 Pluta. R., Januszewski, S. & Czuczwar, S. J. Brain Ischemia as a Prelude to Alzheimer's Disease. Front Aging Neurosci 13, 636653, doi:10.3389/fnagi.2021.636653 (2021).
77 Kalaria, R. N. The role of cerebral ischemia in Alzheimer's disease. Neurobiology of Aging 21, 321-330, doi:https://doi.org/10.1016/S0197-4580(00)00125-1 (2000).
78 Qiao, F. et al. Complement plays an important role in spinal cord injury and represents a therapeutic target for improving recovery following trauma. Am J Pathol 169, 1039-1047, doi:10.2353/ajpath.2006.060248 (2006).
79 Peterson, S. L., Nguyen, H. X., Mendez, O. A. & Anderson, A. J. Complement Protein C3 Suppresses Axon Growth and Promotes Neuron Loss. Scientific Reports 7, 12904, doi:10.1038/s41598-017-11410-x (2017).
80 Wu, T. et al. Complement C3 Is Activated in Human AD Brain and Is Required for Neurodegeneration in Mouse Models of Amyloidosis and Tauopathy. Cell Rep 28, 2111-2123.e2116, doi:10.1016/j.celrep.2019.07.060 (2019).
81 Rodriguez-Jimenez, F. J. et al. Activation of Neurogenesis in Multipotent Stem Cells Cultured In Vitro and in the Spinal Cord Tissue After Severe Injury by Inhibition of Glycogen Synthase Kinase-3. Neurotherapeutics 18, 515-533, doi:10.1007/s13311-020-00928-0 (2021).
82 Renault-Mihara, F. et al. Beneficial compaction of spinal cord lesion by migrating astrocytes through glycogen synthase kinase-3 inhibition. EMBO Mol Med 3, 682-696, doi:10.1002/emmm.201100179 (2011).
83 Hooper, C., Killick, R. & Lovestone, S. The GSK3 hypothesis of Alzheimer's disease. J Neurochem 104, 1433-1439, doi:10.1111/j.1471-4159.2007.05194.x (2008).
84 Lauretti, E., Dincer, O. & Praticò, D. Glycogen synthase kinase-3 signaling in Alzheimer's disease. Biochimica et Biophysica Acta (BBA)—Molecular Cell Research 1867, 118664, doi:https://doi.org/10.1016/j.bbamcr.2020.118664 (2020).
85 Toral-Rios, D., Pichardo-Rojas, P. S., Alonso-Vanegas, M. & Campos-Peia, V. GSK3β and Tau Protein in Alzheimer's Disease and Epilepsy. Frontiers in Cellular Neuroscience 14, doi:10.3389/fncel.2020.00019 (2020).
86 Hu, S. et al. GSK3 inhibitors show benefits in an Alzheimer's disease (AD) model of neurodegeneration but adverse effects in control animals. Neurobiol Dis 33, 193-206, doi:10.1016/j.nbd.2008.10.007 (2009).
87 Wang, H. et al. Tideglusib, a chemical inhibitor of GSK3β, attenuates hypoxic-ischemic brain injury in neonatal mice. Biochim Biophys Acta 1860, 2076-2085. doi:10.1016/j.bbagen.2016.06.027 (2016).
88 Serenò, L. et al. A novel GSK-3beta inhibitor reduces Alzheimer's pathology and rescues neuronal loss in vivo. Neurobiol Dis 35, 359-367, doi:10.1016/j.nbd.2009.05.025 (2009).
89 Lauretti, E., Dincer, O. & Praticò, D. Glycogen synthase kinase-3 signaling in Alzheimer's disease. Biochim Biophys Acta Mol Cell Res 1867, 118664. doi:10.1016/j.bbamcr.2020.118664 (2020).
90 Yang, J. et al. Loss of astrocyte polarization in the tg-ArcSwe mouse model of Alzheimer's disease. J Alzheimers Dis 27, 711-722, doi:10.3233/jad-2011-110725 (2011).
91 Lin, A. et al. Serum amyloid A inhibits astrocyte migration via activating p38 MAPK. Journal of Neuroinflammation 17, 254, doi:10.1186/s12974-020-01924-z (2020).
92 Wei, H. et a. Systematic analysis of purified astrocytes after SCI unveils Zeb2os function during astrogliosis. Cell Rep 34, 108721. doi:10.1016/j.cclrep.2021.108721 (2021).
93 Hsu, J. Y. et al. Matrix metalloproteinase-9 facilitates glial scar formation in the injured spinal cord. J Neurosci 28, 13467-13477, doi:10.1523/jneurosci.2287-08.2008 (2008).
94 Neumaier, F., Zlatopolskiy, B. D. & Neumaier, B. Drug Penetration into the Central Nervous System: Pharmacokinetic Concepts and In Vitro Model Systems. Pharmaceutics 13, doi:10.3390/pharmaceutics13101542 (2021).
95 Chopra, N. et al. Blood-Spinal Cord Barrier: Its Role in Spinal Disorders and Emerging Therapeutic Strategies. NeuroSci 3, 1-27 (2022).
96 Whetstone, W. D., Hsu, J. Y., Eisenberg, M., Werb, Z. & Noble-Haeusslein, L. J. Blood-spinal cord barrier after spinal cord injury: relation to revascularization and wound healing. J Neurosci Res 74, 227-239, doi:10.1002/jnr.10759 (2003).
97 Pan, W., Banks, W. A. & Kastin, A. J. Blood-brain barrier permeability to ebiratide and TNF in acute spinal cord injury. Exp Neurol 146, 367-373, doi:10.1006/exnr.1997.6533 (1997).
98 Cohen, S. J. & Stackman, R. W., Jr. Assessing rodent hippocampal involvement in the novel object recognition task. A review. Behav Brain Res 285, 105-117. doi:10.1016/j.bbr.2014.08.002 (2015).
99 McHail, D. G. & Dumas, T. C. Hippocampal gamma rhythms during Y-maze navigation in the juvenile rat. Hippocampus 30, 505-525, doi:10.1002/hipo.23168 (2020).
100 Samyai, Z. et al. Impaired hippocampal-dependent learning and functional abnormalities in the hippocampus in mice lacking serotonin (1A) receptors. Proc Natl Acad Sci USA 97, 14731-14736, doi:10.1073/pnas.97.26.14731 (2000).
101 Lee, J. K. et al. Assessing spinal axon regeneration and sprouting in Nogo-, MAG-, and OMgp-deficient mice. Neuron 66, 663-670, doi:10.1016/j.neuron.2010.05.002 (2010).
102 Ito, D. et al. Microglia-specific localisation of a novel calcium binding protein, Iba1. Brain Res Mol Brain Res 57, 1-9, doi:10.1016/s0169-328x(98)00040-0 (1998).
103 Hopperton, K. E., Mohammad, D., Trépanier, M. O., Giuliano, V. & Bazinet, R. P. Markers of microglia in post-mortem brain samples from patients with Alzheimer's disease: a systematic review. Molecular Psychiatry 23, 177-198, doi:10.1038/mp.2017.246 (2018).
104 Zhang, D. et al. Microglial activation contributes to cognitive impairments in rotenone-induced mouse Parkinson's disease model. Journal of Neuroinflammation 18, 4, doi:10.1186/s12974-020-02065-z (2021).
105 Lull, M. E. & Block, M. L. Microglial activation and chronic neurodegeneration. Neurotherapeutics 7, 354-365, doi:10.1016/j.nurt.2010.05.014 (2010).
106 Chistiakov, D. A., Killingsworth, M. C., Myasoedova, V. A., Orekhov, A. N. & Bobryshev, Y. V. CD68/macrosialin: not just a histochemical marker. Laboratory Investigation 97, 4-13, doi:10.1038/labinvest.2016.116 (2017).
107 Deng, Y. et al. SOX9 Knockdown-Mediated FOXO3 Downregulation Confers Neuroprotection Against Ischemic Brain Injury. Frontiers in Cell and Developmental Biology 8, doi:10.3389/fcell.2020.555175 (2021).
108 Minett, T. et al. Microglial immunophenotype in dementia with Alzheimer's pathology. J Neuroinflammation 13, 135, doi:10.1186/s12974-016-0601-z (2016).
109 Farso, M., Ménard, C., Colby-Milley, J. & Quirion, R. Immune marker CD68 correlates with cognitive impairment in normally aged rats. Neurobiol Aging 34, 1971-1976, doi:10.1016/j.neurobiolaging.2013.02.008 (2013).
110 Hosseini, A. et al. The local and circulating SOX9 as a potential biomarker for the diagnosis of primary bone cancer. J Bone Oncol 23, 100300, doi:10.1016/j.jbo.2020.100300 (2020).
111 Kawai, T. et al. SOX9 is a novel cancer stem cell marker surrogated by osteopontin in human hepatocellular carcinoma. Scientific Reports 6, 30489, doi:10.1038/srep30489 (2016).
112 Huang, J.-Q. et al. SOX9 drives the epithelial-mesenchymal transition in non-small-cell lung cancer through the Wnt/β-catenin pathway. Journal of Translational Medicine 17, 143, doi:10.1186/s12967-019-1895-2 (2019).
113 Sauerbeck, A. D. et al. Spinal cord injury causes chronic liver pathology in rats. J Neurotrauma 32, 159-169, doi:10.1089/neu.2014.3497 (2015).
114 Goodus, M. T. & McTigue, D. M. Hepatic dysfunction after spinal cord injury: A vicious cycle of central and peripheral pathology? Exp Neurol 325, 113160, doi:10.1016/j.expneurol.2019.113160 (2020).
115 Enoh, J. E., Cho, F. N., Manfo, F. P., Ako, S. E. & Akum, E. A. Abnormal Levels of Liver Enzymes and Hepatotoxicity in HIV-Positive, TB, and HIV/TB-Coinfected Patients on Treatment in Fako Division, Southwest Region of Cameroon. Biomed Res Int 2020, 9631731, doi:10.1155/2020/9631731 (2020).
116 Kim, H. J. et al. Effects of blood urea nitrogen independent of the estimated glomerular filtration rate on the development of anemia in non-dialysis chronic kidney disease: The results of the KNOW-CKD study. PLoS One 16, e0257305, doi:10.1371/journal.pone.0257305 (2021).

1. A method of treating a neurological disorder, injury to the central or peripheral nervous system, and/or secondary pathology thereof, comprising administering to the individual a therapeutically effective amount of a composition comprising a compound of formula I: 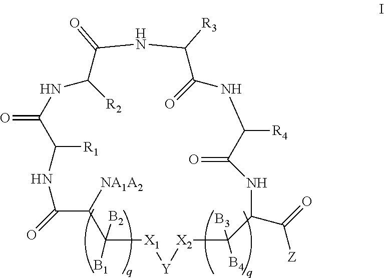
wherein,
A₁ and A₂ are each independently H, alkyl, acyl, heterocyclyl, or aryl;
B₁, B₂, B₃, and B₄ are each independently H or methyl;
X₁ and X₂ are each independently —S—, —O—, —CH₂—, or —NH—;
R₁ is 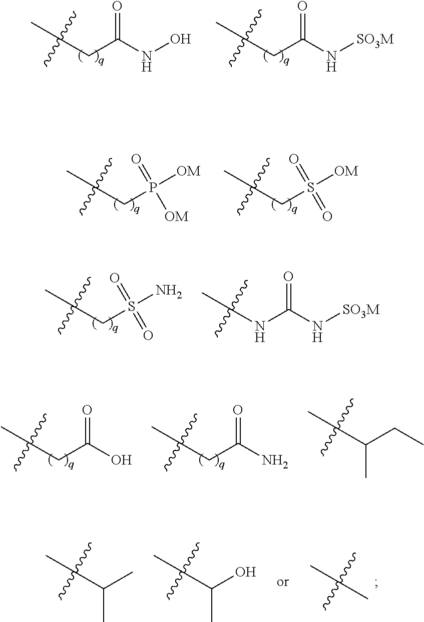
R₂ is 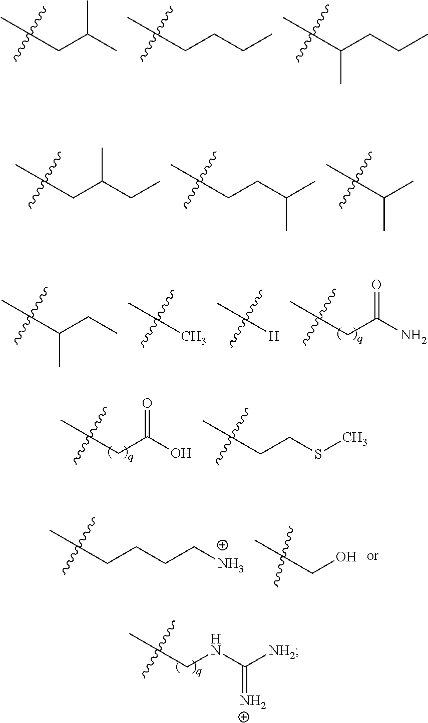
R₃ is 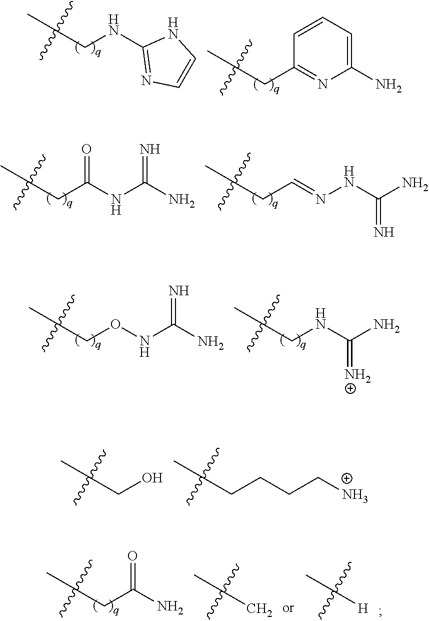
R₄ is 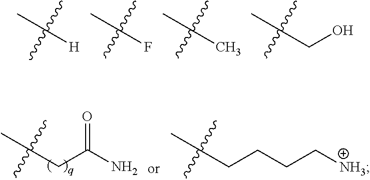
M is H or alkyl;
q is 0, 1, 2, or 3;
Y is a dye or C₄-C₆ alkyl; and
Z is —OH, —O-alkyl, or NA₁A₂, where A₁ and A₂ are as defined above.

2. (canceled)

3. The method of claim 1, wherein the neurological disorder and/or secondary pathology thereof comprises Alzheimer's Disease, multiple sclerosis, frontotemporal dementia, psychiatric disorders, Amyotrophic Lateral Sclerosis (ALS), ataxia, Bell's Palsy, brain tumor, cerebral aneurysm, encephalitis, epilepsy, seizures, Guillain-Barré Syndrome, dementia, migraine, Friedreich ataxia, Huntington's disease, Lewy body disease, Spinal muscular atrophy, or Parkinson's Disease.

4. The method of claim 1, wherein the injury to the central or peripheral nervous system comprises neurotrauma.

5. The method of claim 4, wherein the neurotrauma comprises concussion, traumatic brain injury, skull fracture, spinal column fracture, or spinal cord injury.

6. The method of claim 1, wherein the compound comprises the amino acids DIRG, CDIRGC, or both.

7. (canceled)

8. The method of claim 1, wherein Y is a rhodamine, fluorene, BODIPY® dye, or wherein Y is 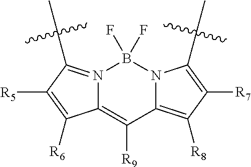
wherein:
R₅, R₆, R₇, and R₈ are each independently H, halide, C₁-C₃ alkyl, or SO₃H or a salt thereof;
R₅ and R₆, can optionally join to form a ring;
R₇ and R₈ can optionally join to form a ring; and
R₉ is H, halide, C₁-C₃ alkyl, or SO₃H or a salt thereof or aryl.

9. (canceled)

10. The method of claim 1, wherein the secondary pathology comprises fibrosis, liver disease, kidney disease, colon disease, a cardiac condition, a metabolic pathology, a muscle-associated disease, respiratory diseases, and/or cancer.

11. (canceled)

12. A method of improving locomotor function, improving motility, improving long-term memory, improving cognition, reducing fibrosis, and/or reducing inflammation in an individual, comprising administering to the individual a composition comprising a compound of formula I: 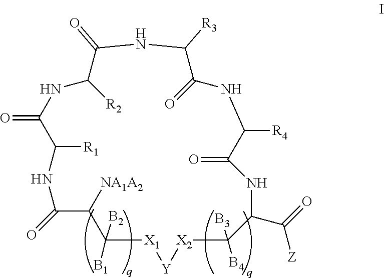
wherein,
A₁ and A₂ are each independently H, alkyl, acyl, heterocyclyl, or aryl;
B₁, B₂, B₃, and B₄ are each independently H or methyl;
X₁ and X₂ are each independently —S—, —O—, —CH₂—, or —NH—;
R₁ is 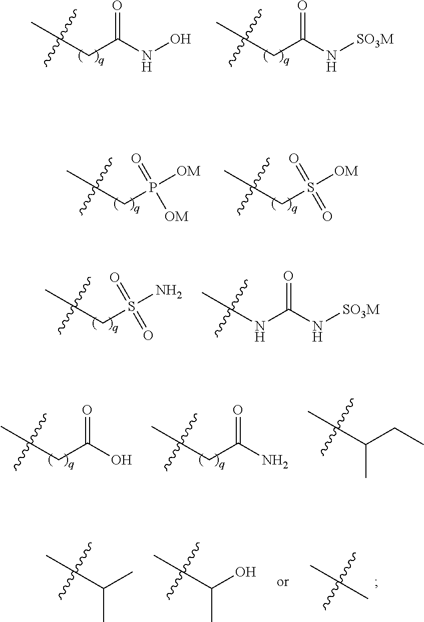
R₂ is 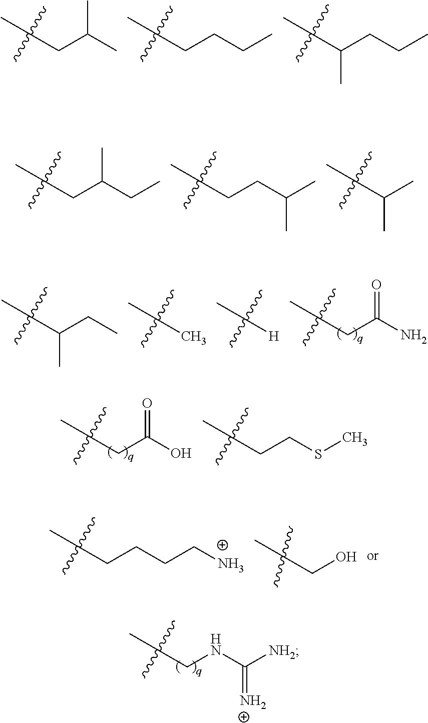
R₃ is 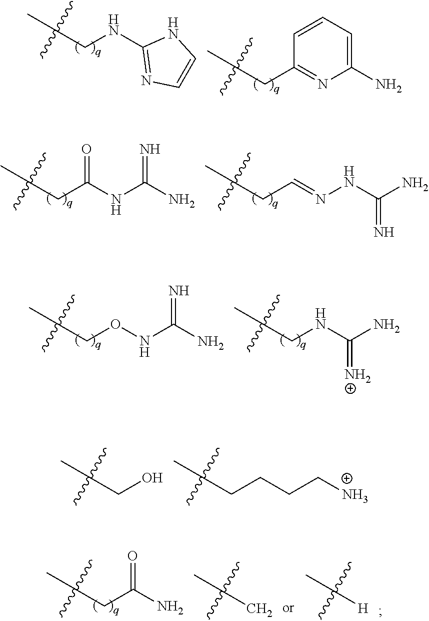
R₄ is 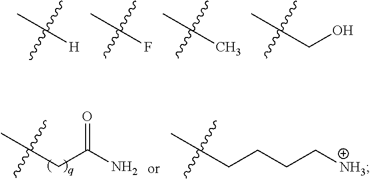
M is H or alkyl;
q is 0, 1, 2, or 3;
Y is a dye or C₄-C₆ alkyl; and
Z is —OH, —O-alkyl, or NA₁A₂, where A₁ and A₂ are as defined above.

13. The method of claim 12, wherein the individual has a neurological disorder, injury to the central or peripheral nervous system, and/or secondary pathology thereof.

14. (canceled)

15. The method of claim 13, wherein the neurological disorder and/or secondary pathology thereof comprises Alzheimer's Disease, multiple sclerosis, frontotemporal dementia, psychiatric disorders, Amyotrophic Lateral Sclerosis (ALS), ataxia, Bell's Palsy, brain tumor, cerebral aneurysm, encephalitis, epilepsy, seizures, Guillain-Barré Syndrome, dementia, migraine, Friedreich ataxia, Huntington's disease, Lewy body disease, Spinal muscular atrophy, or Parkinson's Disease.

16. The method of claim 13, wherein the injury to the central or peripheral nervous system comprises neurotrauma.

17. The method of claim 16, wherein the neurotrauma comprises concussion, traumatic brain injury, skull fracture, spinal column fracture, or spinal cord injury.

18. The method of claim 12, wherein the compound comprises the amino acids DIRG and/or CDIRGC.

19. (canceled)

20. The method of claim 12, wherein Y is a rhodamine, fluorene, BODIPY® dye, or wherein Y is 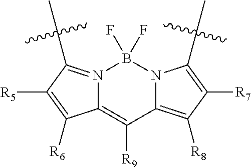
wherein:
R₅, R₆, R₇, and R₈ are each independently H, halide, C₁-C₃ alkyl, or SO₃H or a salt thereof;
R₅ and R₆, can optionally join to form a ring;
R₇ and R₈ can optionally join to form a ring; and
R₉ is H, halide, C₁-C₃ alkyl, or SO₃H or a salt thereof, or aryl.

21. (canceled)

22. The method of claim 12, wherein the secondary pathology comprises fibrosis, liver disease, kidney disease, colon disease, a cardiac condition, a metabolic pathology, a muscle-associated disease, respiratory diseases, and/or cancer.

23. (canceled)

24. A method of monitoring location of a compound in vivo or ex vivo comprising directly or indirectly identifying a compound comprising formula I in the individual and/or a molecule to which the compound binds: 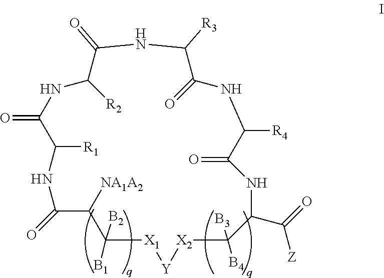
wherein,
A₁ and A₂ are each independently H, alkyl, acyl, heterocyclyl, or aryl;
B₁, B₂, B₃, and B₄ are each independently H or methyl;
X₁ and X₂ are each independently —S—, —O—, —CH₂—, or —NH—;
R₁ is 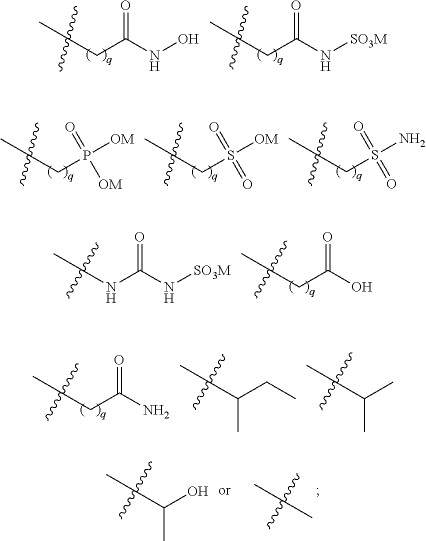
R₂ is 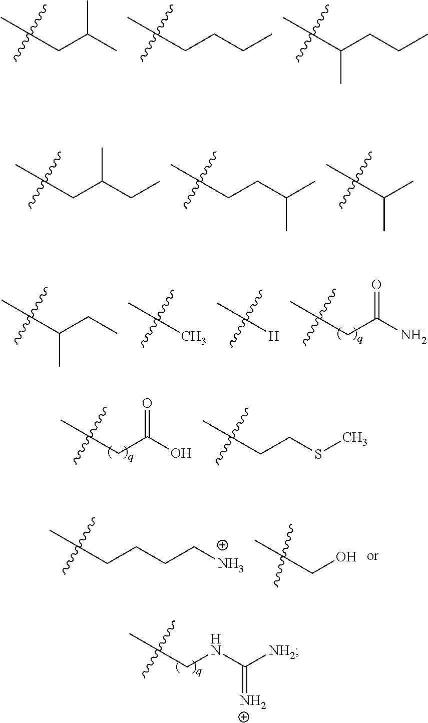
R₃ is 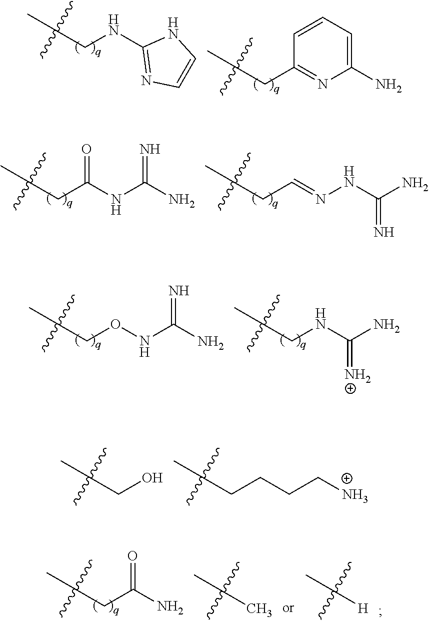
R₄ is 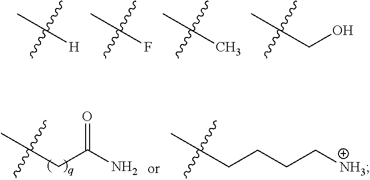
M is H or alkyl;
q is 0, 1, 2, or 3;
Y is a dye or C₄-C₆ alkyl; and
Z is —OH, —O-alkyl, or NA₁A₂, where A₁ and A₂ are as defined above.

25. The method of claim 24, wherein the compound comprises the amino acids DIRG and/or CDIRGC.

26. (canceled)

27. The method of claim 24, wherein Y is a rhodamine, fluorene, BODIPY® dye, or wherein Y is 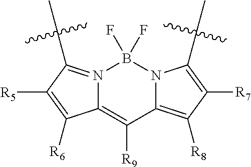
wherein:
R₅, R₆, R₇, and R₈ are each independently H, halide, C₁-C₃ alkyl, or SO₃H or a salt thereof;
R₅ and R₆, can optionally join to form a ring;
R₇ and R₈ can optionally join to form a ring; and
R₉ is H, halide, C₁-C₃ alkyl, or SO₃H or a salt thereof or aryl.

28. (canceled)

29. The method of claim 24, wherein the method: (a) provides information about the mechanism of action, pharmacokinetic, and/or toxicity of the compound;
(b) comprises an immunohistochemical assay; and/or
(c) comprises monitoring in a mammal the location of the compound or a molecule to which it binds.

30. (canceled)

31. (canceled)

32. The method of claim 24, wherein the molecule to which it binds is a cancer antigen or marker.

33. (canceled)

34. (canceled)

35. (canceled)

36. (canceled)

37. (canceled)

38. (canceled)

39. (canceled)

40. (canceled)
